(19) United States
(12) Patent Application Publication
(10) Pub. No.: US 2026/0263214 A1
(43) Pub. Date: Sep. 10, 2026

(54) PROSTHETIC AORTIC VALVE PACING SYSTEMS

(71) Applicant: SMARTVALVES LTD., Savyon (IL)

(72) Inventors: Yossi GROSS, Moshav Mazor (IL); Navot Rabban, Ramat Gan (IL); Meni Iamberger, Kfar Saba (IL); Aharon Daffan, Jerusalem (IL)

(73) Assignee: SMARTVALVES LTD., Savyon (IL)

(21) Appl. No.: 19/466,635

(22) Filed: Feb. 2, 2026

Related U.S. Application Data
(63) Continuation of application No. PCT/IL2024/050830, filed on Aug. 18, 2024.
Application No. PCT/IL2024/050830 is a continuation-in-part of application No. 18/607,638, filed on Mar. 18, 2024.
Application No. 18/607,638 is a continuation-in-part of application No. 18/452,229, filed on Aug. 18, 2023, now Pat. No. 11,931,255.
Application No. 18/452,229 is a continuation-in-part of application No. 18/452,216, filed on Aug. 18, 2023, now Pat. No. 11,975,203.

(51) Int. Cl. A61F 2/24; A61N 1/362; H01Q 1/27

(52) U.S. Cl. CPC A61F 2/2418; A61F 2/2427; A61N 1/3627; H01Q 1/273; A61F 2250/0002; A61F 2250/0058

(57) ABSTRACT

A prosthetic cardiac valve includes a frame and a plurality of prosthetic leaflets coupled to the frame so as to allow blood flow in one direction. An antenna is mechanically coupled to the frame proximal of the prosthetic leaflets, and includes one or more prosthetic-valve coils. First and second proximal peaks respectively defined by circumferentially adjacent first and second proximal-most stent cells of interconnected stent cells of the frame are located at respective first and second peak angular locations about a central longitudinal axis of the frame. The antenna is mechanically coupled to the frame such that a centroid of the antenna is at an antenna angular location between the first and the second peak angular locations, and a proximal-most point of the antenna is axially disposed between 5 mm proximal of and 5 mm distal of the first and the second proximal peaks. Other embodiments are also described.

### CROSS-REFERENCE TO RELATED APPLICATIONS

The present application is a continuation of International Appl. PCT/IL2024/050830, filed Aug. 18, 2024, which published as PCT Publication WO 2025/041129 to Gross et al. and which is a continuation-in-part of U.S. application Ser. No. 18/607,638, filed Mar. 18, 2024, which published as US Patent Application Publication 2025/0058124 to Gross et al. and which (a) is a continuation-in-part of U.S. application Ser. No. 18/452,229, filed Aug. 18, 2023, now U.S. Pat. No. 11,931,255, and (b) is a continuation-in-part of U.S. application Ser. No. 18/452,216, filed Aug. 18, 2023, now U.S. Pat. No. 11,975,203. All of the above-referenced applications are assigned to the assignee of the present application and incorporated herein by reference.

### FIELD OF THE APPLICATION

The present invention relates generally to surgical implants and systems, and specifically to prosthetic aortic valves and systems.

### BACKGROUND OF THE APPLICATION

Aortic heart valve replacement may be necessary to treat valve regurgitation or stenotic calcification of the leaflets. In percutaneous transluminal delivery techniques, a prosthetic aortic valve is compressed for delivery in a catheter and advanced through the descending aorta to the heart, where the prosthetic valve is deployed in the aortic valve annulus. New-onset cardiac conduction disturbances are common after transcatheter aortic valve replacement (TAVR). The most common complication is left bundle branch block (LBBB).

PCT Publication WO 2022/149130 to Gross, which is incorporated herein by reference, inter alia describes a prosthetic aortic valve, which is configured to be delivered to a native aortic valve of a patient in a constrained delivery configuration within a delivery sheath. The prosthetic aortic valve includes a frame, which includes interconnected stent struts arranged so as to define interconnected stent cells; a plurality of prosthetic leaflets coupled to the frame; a cathode and an anode, which are mechanically coupled to the frame; and a prosthetic-valve coil, which is in non-wireless electrical communication with the cathode and the anode, and is coupled to a plurality of the stent struts, running along the stent struts so as to surround a plurality of the stent cells when the prosthetic aortic valve is in an expanded fully-deployed configuration upon release from the delivery sheath.

US Patent Application Publication 2017/0258585 to Marquez et al. describes sensor-integrated prosthetic valves that can comprise a variety of features, including a plurality of valve leaflets, a frame assembly configured to support the plurality of valve leaflets and define a plurality of commissure supports terminating at an outflow end of the prosthetic valve, a sensor device associated with the frame assembly and configured to generate a sensor signal, for example, a sensor signal indicating deflection of one or more of the plurality of commissure supports, and a transmitter assembly configured to receive the sensor signal from the sensor device and wirelessly transmit a transmission signal that is based at least in part on the sensor signal.

U.S. Pat. No. 9,326,854 to Casley et al. describes medical device delivery assemblies. The assembly may include a catheter-based delivery system. The assembly may include a pacing element to pace a patient's heart before, during, or after a procedure. The pacing element may be a detachable, implanting pacing element. The pacing element may be an implantable pacemaker and the implantable pacemaker may be disposed on a catheter-based delivery system. The assembly may include a prosthetic heart valve with one or more pacing elements on it. The pacing element may include a pacing strip or strips. These strips may be conductive or insulative. These strips may prevent, treat, or correct abnormal electrical communication in a heart.

### SUMMARY OF THE APPLICATION

Some embodiments of the present invention provide a prosthetic aortic valve, which is configured to be implanted in a native aortic valve of a patient, and which comprises a plurality of prosthetic leaflets, a frame, and one or more electrodes, including a cathode and an anode, mechanically coupled to the frame. The prosthetic aortic valve further comprises a prosthetic-valve coil, which is in non-wireless electrical communication with the cathode and the anode.

For some applications, the prosthetic aortic valve further comprises circuitry, which is configured to apply pacing to the heart using the one or more electrodes. For example, the pacing may be applied temporarily for up to several weeks after implantation of the prosthetic aortic valve, typically using an external control unit to continuously provide power, or applied longer-term, in which case the prosthetic aortic valve may further comprise an energy storage module, e.g., comprising a battery, which may be periodically charged using the external control unit. Further alternatively or additionally, for some applications, the circuitry is configured to apply rapid pacing during an invasive structural heart procedure, such as an implantation procedure, such as a transcatheter aortic valve replacement (TAVR)-in-TAVR procedure in which the first TAVR comprises the prosthetic aortic valve.

The following Inventive Concepts are therefore provided in accordance with respective applications of the present invention:

Inventive Concept 1. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction; and
an antenna, which is mechanically coupled to the frame proximal of the prosthetic leaflets, and which comprises one or more prosthetic-valve coils,
wherein first and second proximal peaks respectively defined by circumferentially adjacent first and second proximal-most stent cells of the interconnected stent cells are located at respective first and second peak angular locations about the central longitudinal axis of the frame, and
wherein the antenna is mechanically coupled to the frame such that (a) a centroid of the antenna is at an antenna angular location about the central longitudinal axis, the antenna angular location between the first and the second peak angular locations, and (b) a proximal-most point of the antenna is axially disposed between (i) 5 mm proximal of the first and the second proximal peaks and (ii) 5 mm distal of the first and the second proximal peaks.

Inventive Concept 2. The prosthetic cardiac valve according to Inventive Concept 1, wherein the antenna is mechanically coupled to the frame such that the proximal-most point of the antenna is axially disposed between (i) 3 mm proximal of the first and the second proximal peaks and (ii) 5 mm distal of the first and the second proximal peaks.

Inventive Concept 3. The prosthetic cardiac valve according to any one of Inventive Concepts 1-2,
wherein the prosthetic cardiac valve is a prosthetic aortic valve,
wherein the antenna is mechanically coupled to the frame downstream of the prosthetic leaflets,
wherein the circumferentially adjacent first and second proximal-most stent cells are circumferentially adjacent first and second downstream-most stent cells, respectively,
wherein the first and the second proximal peaks are first and second downstream peaks respectively defined by the circumferentially adjacent first and second downstream-most stent cells, and
wherein the proximal-most point of the antenna is a downstream-most point of the antenna that is axially disposed between (i) 5 mm downstream of the first and the second downstream peaks and (ii) 5 mm upstream of the first and the second downstream peaks.

Inventive Concept 4. The prosthetic cardiac valve according to any one of Inventive Concepts 1-2,
wherein the prosthetic cardiac valve is a prosthetic atrioventricular valve,
wherein the antenna is mechanically coupled to the frame upstream of the prosthetic leaflets,
wherein the circumferentially adjacent first and second proximal-most stent cells are circumferentially adjacent first and second upstream-most stent cells, respectively,
wherein the first and the second proximal peaks are first and second upstream peaks respectively defined by the circumferentially adjacent first and second upstream-most stent cells, and
wherein the proximal-most point of the antenna is an upstream-most point of the antenna that is axially disposed between (i) 5 mm upstream of the first and the second upstream peaks and (ii) 5 mm downstream of the first and the second upstream peaks.

Inventive Concept 5. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises:
   interconnected stent struts arranged so as to define interconnected stent cells; and
   one or more delivery-tool-coupling tabs, disposed proximal of the stent cells, and shaped so as to define respective distal-facing edges;
a plurality of prosthetic leaflets coupled to the frame so as to allow blood flow in a downstream direction and inhibit blood flow in an upstream direction; and
an antenna, which is mechanically coupled to the frame proximal of the prosthetic leaflets, and which comprises one or more prosthetic-valve coils,
wherein first and second proximal peaks respectively defined by circumferentially adjacent first and second proximal-most stent cells of the interconnected stent cells are located at respective first and second peak angular locations about the central longitudinal axis of the frame, and
wherein the antenna is mechanically coupled to the frame such that (a) a centroid of the antenna is at an antenna angular location about the central longitudinal axis, the antenna angular location between the first and the second peak angular locations, and (b) a proximal-most point of the antenna is axially disposed between (i) an axial position of the distal-facing edges of the delivery-tool-coupling tabs and (ii) 5 mm distal of the first and the second proximal peaks.

Inventive Concept 6. A valve prosthesis system comprising the prosthetic cardiac valve according to Inventive Concept 5, the valve prosthesis system further comprising a delivery system, which comprises a delivery shaft that is removably couplable to the one or more delivery-tool-coupling tabs.

Inventive Concept 7. The prosthetic cardiac valve according to Inventive Concept 5,
wherein the prosthetic cardiac valve is a prosthetic aortic valve,
wherein the antenna is mechanically coupled to the frame downstream of the prosthetic leaflets,
wherein the one or more delivery-tool-coupling tabs are disposed downstream of the stent cells, and shaped so as to define respective upstream-facing edges,
wherein the circumferentially adjacent first and second proximal-most stent cells are circumferentially adjacent first and second downstream-most stent cells, respectively,
wherein the first and the second proximal peaks are first and second downstream peaks respectively defined by the circumferentially adjacent first and second downstream-most stent cells, and
wherein the proximal-most point of the antenna is a downstream-most point of the antenna that is axially disposed between (i) an axial position of the upstream-facing edges of the delivery-tool-coupling tabs and (ii) 5 mm upstream of the first and the second downstream peaks.

Inventive Concept 8. The prosthetic cardiac valve according to Inventive Concept 5,
wherein the prosthetic cardiac valve is a prosthetic atrioventricular valve,
wherein the antenna is mechanically coupled to the frame upstream of the prosthetic leaflets,
wherein the one or more delivery-tool-coupling tabs are disposed upstream of the stent cells, and shaped so as to define respective downstream-facing edges,
wherein the circumferentially adjacent first and second proximal-most stent cells are circumferentially adjacent first and second upstream-most stent cells, respectively,
wherein the first and the second proximal peaks are first and second upstream peaks respectively defined by the circumferentially adjacent first and second upstream-most stent cells, and
wherein the proximal-most point of the antenna is an upstream-most point of the antenna that is axially disposed between (i) an axial position of the downstream-facing edges of the delivery-tool-coupling tabs and (ii) 5 mm downstream of the first and the second upstream peaks.

Inventive Concept 9. The prosthetic cardiac valve according to any one of Inventive Concepts 1 and 5, wherein the antenna comprises a magnetic core around which are wound the one or more prosthetic-valve coils.

Inventive Concept 10. The prosthetic cardiac valve according to any one of Inventive Concepts 1 and 5,
wherein the first and the second proximal-most stent cells are joined at a cell junction,
wherein the first proximal-most stent cell comprises a right proximal strut of the interconnected stent struts, the right proximal strut extending between the cell junction and a first proximal peak defined by the first proximal-most stent cell,
wherein the second proximal-most stent cell comprises a left proximal strut of the interconnected stent struts, the left proximal strut extending between the cell junction and a second proximal peak defined by the second proximal-most stent cell,
wherein a flexible sheet is mechanically coupled to the right and the left proximal struts, and
wherein the antenna is mechanically coupled to the frame at least in part by being mechanically coupled to the flexible sheet between the right and the left proximal struts.

Inventive Concept 11. The prosthetic cardiac valve according to any one of Inventive Concepts 1 and 5, wherein the first and the second proximal-most stent cells are joined at a cell junction, and the antenna is mechanically coupled to the frame at least in part by being mechanically coupled to the cell junction.

Inventive Concept 12. The prosthetic cardiac valve according to Inventive Concept 11, wherein a distal-most point of the antenna coincides with, or is no more than a distance distal of, the cell junction, the distance equal to 30% of a length of the antenna, the distance and the length measured parallel to the central longitudinal axis of the frame.

Inventive Concept 13. The prosthetic cardiac valve according to any one of Inventive Concepts 1 and 5,
wherein the first and the second peak angular locations are angularly offset by a peak-to-peak angular offset,
wherein the first peak angular location and the antenna angular location are angularly offset by a peak-to-antenna angular offset, and
wherein the peak-to-antenna angular offset equals 25%-75% of the peak-to-peak angular offset.

Inventive Concept 14. The prosthetic cardiac valve according to any one of Inventive Concepts 1 and 5, wherein the proximal-most point of the antenna is axially disposed between 5 mm proximal of the first and the second proximal peaks and 5 mm distal of the first and the second proximal peaks.

Inventive Concept 15. The prosthetic cardiac valve according to any one of Inventive Concepts 1 and 5,
wherein the circumferentially adjacent first and second proximal-most stent cells are joined at a cell junction,
wherein a peak height equals a distance between a proximal-most point of the first proximal peak and the cell junction, measured parallel to the central longitudinal axis of the frame, and
wherein a length of the antenna equals 30%-150% of the peak height, the length and the peak height measured parallel to the central longitudinal axis of the frame.

Inventive Concept 16. The prosthetic cardiac valve according to any one of Inventive Concepts 1 and 5,
wherein the first and the second peak angular locations are angularly offset by a peak-to-peak angular offset, and
wherein a width of the antenna, measured in a peak-to-peak direction, equals 10%-60% of the peak-to-peak angular offset.

Inventive Concept 17. The prosthetic cardiac valve according to any one of Inventive Concepts 1 and 5, further comprising:
a cathode and an anode, which are mechanically coupled to the frame; and
circuitry, which is electrically coupled to the cathode, the anode, and the one or more prosthetic-valve coils.

Inventive Concept 18. A valve prosthesis system comprising the prosthetic cardiac valve according to any one of Inventive Concepts 1 and 5, the valve prosthesis system further comprising an external unit, wherein the external unit is configured to be disposed outside a body of the patient, and comprises:
an energy-transmission coil; and
external-unit control circuitry, which is configured to drive the energy-transmission coil to wirelessly transfer energy to at least one of the one or more prosthetic-valve coils by inductive coupling.

Inventive Concept 19. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction;
an antenna, which is mechanically coupled to the frame proximal of the prosthetic leaflets, and which comprises one or more prosthetic-valve coils; and
a flexible sheet,
wherein circumferentially adjacent first and second proximal-most stent cells of the interconnected stent cells are joined at a cell junction,
wherein the first proximal-most stent cell comprises a right proximal strut of the interconnected stent struts, the right proximal strut extending between the cell junction and a first proximal peak defined by the first proximal-most stent cell,
wherein the second proximal-most stent cell comprises a left proximal strut of the interconnected stent struts, the left proximal strut extending between the cell junction and a second proximal peak defined by the second proximal-most stent cell,
wherein the flexible sheet is mechanically coupled to the right and the left proximal struts, and
wherein the antenna is mechanically coupled to the frame at least in part by being mechanically coupled to the flexible sheet between the right and the left proximal struts.

Inventive Concept 20. The prosthetic cardiac valve according to Inventive Concept 19, wherein the antenna is mechanically coupled to the frame at least in part by being mechanically coupled to the cell junction.

Inventive Concept 21. The prosthetic cardiac valve according to Inventive Concept 19, wherein the antenna is mechanically coupled to the flexible sheet by stitching.

Inventive Concept 22. The prosthetic cardiac valve according to Inventive Concept 19, wherein the flexible sheet is mechanically coupled to the right and the left proximal struts by stitching.

Inventive Concept 23. The prosthetic cardiac valve according to Inventive Concept 19, wherein the antenna comprises a magnetic core around which are wound the one or more coils.

Inventive Concept 24. The prosthetic cardiac valve according to Inventive Concept 19, wherein the flexible sheet is coupled only to one or more of the interconnected stent struts of each of the first and the second proximal-most stent cells, and not to any of the interconnected stent struts of other stent cells of the frame.

Inventive Concept 25. The prosthetic cardiac valve according to any one of Inventive Concepts 19-24, wherein the frame further comprises one or more delivery-tool-coupling tabs, disposed proximal of the stent cells.

Inventive Concept 26. A valve prosthesis system comprising the prosthetic cardiac valve according to any one of Inventive Concepts 19-25, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

Inventive Concept 27. The prosthetic cardiac valve according to any one of Inventive Concepts 19-25,
wherein the prosthetic cardiac valve is a prosthetic aortic valve,
wherein the antenna is mechanically coupled to the frame downstream of the prosthetic leaflets,
wherein the circumferentially adjacent first and second proximal-most stent cells are circumferentially adjacent first and second downstream-most stent cells,
wherein the right proximal strut is a right downstream strut of the interconnected stent struts,
wherein the first proximal peak defined by the first proximal-most stent cell is a first downstream peak defined by the first downstream-most stent cell,
wherein the left proximal strut is a left downstream strut of the interconnected stent struts,
wherein the second proximal peak defined by the second proximal-most stent cell is s second downstream peak defined by the second downstream-most stent cell,
wherein the flexible sheet is mechanically coupled to the right and the left downstream struts, and
wherein the antenna is mechanically coupled to the frame at least in part by being mechanically coupled to the flexible sheet between the right and the left downstream struts.

Inventive Concept 28. The prosthetic cardiac valve according to any one of Inventive Concepts 19-25,
wherein the prosthetic cardiac valve is a prosthetic atrioventricular valve,
wherein the antenna is mechanically coupled to the frame upstream of the prosthetic leaflets,
wherein the circumferentially adjacent first and second proximal-most stent cells are circumferentially adjacent first and second upstream-most stent cells,
wherein the right proximal strut is a right upstream strut of the interconnected stent struts,
wherein the first proximal peak defined by the first proximal-most stent cell is a first upstream peak defined by the first upstream-most stent cell,
wherein the left proximal strut is a left upstream strut of the interconnected stent struts,
wherein the second proximal peak defined by the second proximal-most stent cell is s second upstream peak defined by the second upstream-most stent cell,
wherein the flexible sheet is mechanically coupled to the right and the left upstream struts, and
wherein the antenna is mechanically coupled to the frame at least in part by being mechanically coupled to the flexible sheet between the right and the left upstream struts.

Inventive Concept 29. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells, wherein distal ones of the stent cells are located in a distal half of the frame and define respective distal peaks;
a plurality of prosthetic leaflets coupled to the frame so as to allow blood flow in a downstream direction and inhibit blood flow in an upstream direction;
an electrode, which is disposed at or near a distal peak of one of the distal stent cells, wherein first and second distal stent struts of the one of the distal stent cells are joined at the distal peak; and
coupling material, which is shaped so as to define:
   (a) a first strip that is mechanically coupled to the first distal stent strut,
   (b) a second strip that is mechanically coupled to the second distal stent strut, and
   (c) a junction, which couples together the first and the second strips,
   such that the first and the second strips together couple the electrode to the frame at or near the distal peak.

Inventive Concept 30. The prosthetic cardiac valve according to Inventive Concept 29, wherein the distal ones of the stent cells are distal-most ones of the stent cells, and the one of the distal stent cells is one of the distal-most stent cells.

Inventive Concept 31. The prosthetic cardiac valve according to Inventive Concept 29, wherein the first and the second strips are mechanically coupled to the first and the second distal stent struts, respectively, by stitching.

Inventive Concept 32. The prosthetic cardiac valve according to Inventive Concept 29, wherein the junction of the coupling material is mechanically coupled to the frame at or near the distal peak.

Inventive Concept 33. The prosthetic cardiac valve according to Inventive Concept 29, wherein the first strip has length equal to at least 50% of a length of the first distal stent strut.

Inventive Concept 34. The prosthetic cardiac valve according to Inventive Concept 33, wherein the length of the first strip is greater than the length of the first distal stent strut.

Inventive Concept 35. The prosthetic cardiac valve according to Inventive Concept 29, wherein the second strip has length equal to at least 50% of a length of the second distal stent strut.

Inventive Concept 36. The prosthetic cardiac valve according to Inventive Concept 35, wherein the length of the second strip is no more than 100% of the length of the second distal stent strut.

Inventive Concept 37. The prosthetic cardiac valve according to any one of Inventive Concepts 29-36,
wherein the one of the distal stent cells is a first one of the distal stent cells,
wherein the first one of the distal stent cells is joined at a cell junction to a circumferentially-adjacent second one of the distal stent cells, and
wherein the second strip is mechanically coupled to the cell junction.

Inventive Concept 38. The prosthetic cardiac valve according to Inventive Concept 37, wherein the second strip is mechanically coupled to the cell junction by stitching.

Inventive Concept 39. The prosthetic cardiac valve according to any one of Inventive Concepts 29-36,
wherein the prosthetic cardiac valve further comprises an electrical lead, which is electrically coupled to the electrode, and
wherein the first strip is mechanically coupled to at least a portion of the electrical lead.

Inventive Concept 40. The prosthetic cardiac valve according to Inventive Concept 39,
wherein the first strip comprises electrical insulation, and
wherein the first strip electrically insulates the at least a portion of the electrical lead.

Inventive Concept 41. The prosthetic cardiac valve according to Inventive Concept 40, wherein the first strip comprises an elongate portion of a printed circuit board (PCB) with which the electrical lead is integral.

Inventive Concept 42. The prosthetic cardiac valve according to Inventive Concept 39, further comprising circuitry, which is electrically coupled to the electrode by the electrical lead.

Inventive Concept 43. The prosthetic cardiac valve according to any one of Inventive Concepts 29-36,
wherein the first and the second strips are outer first and second strips, which are mechanically coupled to radially outer sides of the first and the second distal stent struts, respectively,
wherein the coupling material is shaped so as to further define:
   (a) an inner first strip that is mechanically coupled to a radially inner side of the first distal stent strut, and
   (b) an inner second strip that is mechanically coupled to a radially inner side of the second distal stent strut,
wherein the junction of the coupling material couples together the outer first strip, the outer second strip, the inner first strip, and the inner second strip, and
wherein the outer first strip, the outer second strip, the inner first strip, and the inner second strip together couple the electrode to the frame at or near the distal peak.

Inventive Concept 44. The prosthetic cardiac valve according to Inventive Concept 43, wherein the junction of the coupling material is folded over the distal peak.

Inventive Concept 45. The prosthetic cardiac valve according to Inventive Concept 44, wherein the folded junction is mechanically coupled to the frame at or near the distal peak.

Inventive Concept 46. The prosthetic cardiac valve according to any one of Inventive Concepts 29-45, wherein the frame further comprises one or more delivery-tool-coupling tabs, disposed proximal of the stent cells.

Inventive Concept 47. A valve prosthesis system comprising the prosthetic cardiac valve according to any one of Inventive Concepts 29-46, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

Inventive Concept 48. The prosthetic cardiac valve according to any one of Inventive Concepts 29-46,
wherein the prosthetic cardiac valve is a prosthetic aortic valve,
wherein the distal half of the frame is an upstream half of the frame,
wherein the distal peaks are upstream peaks,
wherein the distal ones of the stent cells are upstream ones of the stent cells that are located in the upstream half of the frame and define the respective upstream peaks,
wherein the distal peak of the one of the distal stent cells is an upstream peak of one of the upstream stent cells, and
wherein the first and the second distal stent struts of the one of the distal stent cells are first and second upstream stent struts of the one of the upstream stent cells, which are joined at the upstream peak.

Inventive Concept 49. The prosthetic cardiac valve according to any one of Inventive Concepts 29-46,
wherein the prosthetic cardiac valve is a prosthetic atrioventricular valve,
wherein the distal half of the frame is a downstream half of the frame,
wherein the distal peaks are downstream peaks,
wherein the distal ones of the stent cells are downstream ones of the stent cells that are located in the downstream half of the frame and define the respective downstream peaks,
wherein the distal peak of the one of the distal stent cells is a downstream peak of one of the downstream stent cells, and
wherein the first and the second distal stent struts of the one of the distal stent cells are first and second downstream stent struts of the one of the downstream stent cells, which are joined at the downstream peak.

Inventive Concept 50. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells, including a first stent cell shaped so as to define:
   two peaks, consisting of a distal peak and a proximal peak,
   two lateral nodes, consisting of a left lateral node and a right lateral node,
   two left stent struts, consisting of (a) a distal left stent strut joined with the distal peak and the left lateral node, and (b) a proximal left stent strut joined with the proximal peak and the left lateral node, and
   two right stent struts, consisting of (a) a distal right stent strut joined with the distal peak and the right lateral node, and (b) a proximal right stent strut joined with the proximal peak and the right lateral node;
a plurality of prosthetic leaflets coupled to the frame so as to allow blood flow in a downstream direction and inhibit blood flow in an upstream direction;
an electronic component, which is disposed at or near one of the peaks; and
coupling material, which is shaped so as to define:
   (a) a first strip that is mechanically coupled to at least one of the left stent struts,
   (b) a second strip that is mechanically coupled to at least one of the right stent struts, and
   (c) a junction, which couples together the first and the second strips,
   such that the first and the second strips together couple the electronic component to the frame at or near the one of the peaks.

Inventive Concept 51. The prosthetic cardiac valve according to Inventive Concept 50, wherein the prosthetic cardiac valve comprises circuitry, which comprises the electronic component, and which is disposed at or near the one of the peaks.

Inventive Concept 52. The prosthetic cardiac valve according to Inventive Concept 50, wherein the electronic component comprises an electrode.

Inventive Concept 53. The prosthetic cardiac valve according to Inventive Concept 50, wherein the electronic component comprises an energy storage module.

Inventive Concept 54. The prosthetic cardiac valve according to Inventive Concept 50, wherein the first and the second strips together couple the electronic component to the frame at least partially outside the first stent cell at or near the one of the peaks.

Inventive Concept 55. The prosthetic cardiac valve according to Inventive Concept 50, wherein the first and the second strips are mechanically coupled to the at least one of the left stent struts and the at least one of the right stent struts, respectively, by stitching.

Inventive Concept 56. The prosthetic cardiac valve according to Inventive Concept 50, wherein the junction of the coupling material is mechanically coupled to the frame at or near the one of the peaks.

Inventive Concept 57. The prosthetic cardiac valve according to Inventive Concept 50, wherein the first strip has length equal to at least 50% of a length of the at least one of the left stent struts.

Inventive Concept 58. The prosthetic cardiac valve according to Inventive Concept 57, wherein the length of the first strip is greater than the length of the at least one of the left stent struts.

Inventive Concept 59. The prosthetic cardiac valve according to Inventive Concept 50, wherein the second strip has length equal to at least 50% of a length of the at least one of the right stent struts.

Inventive Concept 60. The prosthetic cardiac valve according to Inventive Concept 59, wherein the length of the second strip is greater than the length of the at least one of the right stent struts.

Inventive Concept 61. The prosthetic cardiac valve according to Inventive Concept 50, wherein the first strip is mechanically coupled to the left lateral node.

Inventive Concept 62. The prosthetic cardiac valve according to Inventive Concept 61, wherein the first strip is mechanically coupled to the left lateral node by stitching.

Inventive Concept 63. The prosthetic cardiac valve according to Inventive Concept 50, wherein the second strip is mechanically coupled to the right lateral node.

Inventive Concept 64. The prosthetic cardiac valve according to Inventive Concept 63, wherein the second strip is mechanically coupled to the right lateral node by stitching.

Inventive Concept 65. The prosthetic cardiac valve according to any one of Inventive Concepts 50-64,
wherein the prosthetic cardiac valve further comprises an electrical lead, which is electrically coupled to the electronic component, and
wherein the first strip is mechanically coupled to at least a portion of the electrical lead.

Inventive Concept 66. The prosthetic cardiac valve according to Inventive Concept 65,
wherein the first strip comprises electrical insulation, and
wherein the first strip electrically insulates the at least a portion of the electrical lead.

Inventive Concept 67. The prosthetic cardiac valve according to Inventive Concept 66, wherein the first strip comprises an elongate portion of a printed circuit board (PCB) with which the electrical lead is integral.

Inventive Concept 68. The prosthetic cardiac valve according to any one of Inventive Concepts 50-67, wherein the frame further comprises one or more delivery-tool-coupling tabs, disposed proximal of the stent cells.

Inventive Concept 69. A valve prosthesis system comprising the prosthetic cardiac valve according to any one of Inventive Concepts 50-68, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

Inventive Concept 70. The prosthetic cardiac valve according to any one of Inventive Concepts 50-68,
wherein the prosthetic cardiac valve is a prosthetic aortic valve,
wherein the distal peak and the proximal peak are an upstream peak and a downstream peak, respectively,
wherein the distal left stent strut is an upstream left stent strut,
wherein the proximal left stent strut is a downstream left stent strut,
wherein the distal right stent strut is an upstream right stent strut, and
wherein the proximal right stent strut is a downstream right stent strut.

Inventive Concept 71. The prosthetic cardiac valve according to any one of Inventive Concepts 50-68,
wherein the prosthetic cardiac valve is a prosthetic atrioventricular valve,
wherein the distal peak and the proximal peak are a downstream peak and an upstream peak, respectively,
wherein the distal left stent strut is a downstream left stent strut,
wherein the proximal left stent strut is an upstream left stent strut,
wherein the distal right stent strut is a downstream right stent strut, and
wherein the proximal right stent strut is an upstream right stent strut.

Inventive Concept 72. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction when the prosthetic cardiac valve is in the expanded deployment configuration;
circuitry, which is mechanically coupled to the frame;
an electrode, which is mechanically coupled to the frame;
a printed circuit board (PCB), which is shaped so as to define an elongate portion; and
an electrical lead, which electrically couples the electrode to the circuitry, and which is integral with the elongate portion of the PCB,
wherein the elongate portion of the PCB is mechanically coupled to some of the interconnected stent struts of the frame.

Inventive Concept 73. The prosthetic cardiac valve according to Inventive Concept 72, wherein the electrical lead is encased in the elongate portion of the PCB.

Inventive Concept 74. The prosthetic cardiac valve according to Inventive Concept 72, wherein the elongate portion of the PCB has an undulating shape that generally runs along the interconnected stent struts.

Inventive Concept 75. The prosthetic cardiac valve according to Inventive Concept 72, wherein the elongate portion of the PCB is shaped so as to follow a path of the interconnected stent struts.

Inventive Concept 76. The prosthetic cardiac valve according to Inventive Concept 72, wherein the elongate portion of the PCB has a same general shape as the interconnected stent struts.

Inventive Concept 77. The prosthetic cardiac valve according to Inventive Concept 72, wherein a length of the elongate portion of the PCB, measured in a straight line between endpoints of the elongate portion, equals 50%-100% of a length of the frame, measured parallel to the central longitudinal axis of the frame.

Inventive Concept 78. The prosthetic cardiac valve according to Inventive Concept 72, wherein a length of the elongate portion of the PCB, measured in a straight line between endpoints of the elongate portion, equals 150%-1000% of a greatest dimension of the circuitry portion of the PCB.

Inventive Concept 79. The prosthetic cardiac valve according to Inventive Concept 72, wherein a length of the elongate portion of the PCB, measured in a straight line between endpoints of the elongate portion, is 0.5-6 cm.

Inventive Concept 80. The prosthetic cardiac valve according to Inventive Concept 72, wherein a portion of the elongate portion between the circuitry portion of the PCB and the electrode has a length equal to 50%-100% of a length of the frame, measured parallel to the central longitudinal axis of the frame.

Inventive Concept 81. The prosthetic cardiac valve according to Inventive Concept 72, wherein a portion of the elongate portion between the circuitry portion of the PCB and the electrode has a length equal to 150%-1000% of a greatest dimension of the circuitry portion of the PCB.

Inventive Concept 82. The prosthetic cardiac valve according to Inventive Concept 72, wherein a portion of the elongate portion between the circuitry portion of the PCB and the electrode has a length of 0.5-6 cm.

Inventive Concept 83. The prosthetic cardiac valve according to Inventive Concept 72, wherein the elongate portion of the PCB has a width, perpendicular to a thickness of the PCB, of 0.4-1.5 mm.

Inventive Concept 84. The prosthetic cardiac valve according to Inventive Concept 72, wherein the elongate portion of the PCB has a width, perpendicular to a thickness of the PCB, equal to 20%-120% of a shortest dimension of the circuitry portion of the PCB perpendicular to a thickness of the circuitry portion.

Inventive Concept 85. The prosthetic cardiac valve according to Inventive Concept 72, wherein the electrode is mechanically coupled to the frame at or near a distal peak of one of distal-most ones of the stent cells.

Inventive Concept 86. The prosthetic cardiac valve according to Inventive Concept 72, wherein the stent struts and the elongate portion of the PCB are rectangular in cross section taken perpendicular to respective longitudinal axes of the stent struts and the elongate portion.

Inventive Concept 87. The prosthetic cardiac valve according to Inventive Concept 72, wherein a ratio of a thickness of the stent struts to a thickness of the electrical lead is 5-15.

Inventive Concept 88. The prosthetic cardiac valve according to Inventive Concept 72, wherein a ratio of a thickness of the stent struts to a thickness of the elongate portion of the PCB is 2-5.

Inventive Concept 89. The prosthetic cardiac valve according to any one of Inventive Concepts 72-88, wherein the circuitry comprises (a) a circuitry portion of the PCB distinct from the elongate portion of the PCB, (b) tracks of the PCB, (c) conductive pads of the PCB, and (d) electronic components coupled to the PCB.

Inventive Concept 90. The prosthetic cardiac valve according to Inventive Concept 89,
wherein the circuitry portion of the PCB is a first circuitry portion of the PCB, and
wherein the PCB is shaped so as to define:
   a second circuitry portion, comprising one or more electronic components, and
   an elongate circuitry-connecting portion, which connects the first circuitry portion to the second circuitry portion, and which comprises an electrical lead that is integral with the elongate circuitry-connecting portion.

Inventive Concept 91. The prosthetic cardiac valve according to Inventive Concept 90, wherein the elongate circuitry-connecting portion is oriented circumferentially around a circumferential portion of the frame.

Inventive Concept 92. The prosthetic cardiac valve according to Inventive Concept 90, wherein the one or more electronic components of the second circuitry portion comprise an energy storage module.

Inventive Concept 93. The prosthetic cardiac valve according to Inventive Concept 89, wherein the circuitry portion of the PCB is an end portion of the PCB.

Inventive Concept 94. The prosthetic cardiac valve according to Inventive Concept 89, wherein the elongate portion of the PCB extends directly from the circuitry portion of the PCB.

Inventive Concept 95. The prosthetic cardiac valve according to Inventive Concept 89, wherein the elongate portion of the PCB is integral with the circuitry portion the PCB.

Inventive Concept 96. The prosthetic cardiac valve according to Inventive Concept 95, wherein the electrical lead is fabricated as a track of the elongate portion of the PCB in connection with one or more of the tracks of the PCB are that part of the circuitry.

Inventive Concept 97. The prosthetic cardiac valve according to any one of Inventive Concepts 72-88,
wherein the elongate portion of the PCB is mechanically coupled to some of the interconnected stent struts of the frame by suturing using sutures, and
wherein the elongate portion of the PCB is shaped so as to define a plurality of protrusions along the elongate portion, which inhibit the sutures from sliding along the elongate portion, such that the sutures fix the elongate portion of the PCB securely to the stent struts.

Inventive Concept 98. The prosthetic cardiac valve according to Inventive Concept 97, wherein the protrusions protrude laterally from the elongate portion of the PCB in a plane defined by the PCB.

Inventive Concept 99. The prosthetic cardiac valve according to Inventive Concept 98, wherein an average distance of lateral protrusion of the protrusions beyond non-protruding portions of the elongate portion, in a single direction, equals 20%-100% of widths of the elongate portion of the PCB at respective locations of the protrusions along the elongate portion, the average distance and the widths measured in the plane defined by the PCB.

Inventive Concept 100. The prosthetic cardiac valve according to any one of Inventive Concepts 72-88, wherein the elongate portion of the PCB is bifurcated, so as to define a main elongate portion and two or more bifurcation elongate portions.

Inventive Concept 101. The prosthetic cardiac valve according to Inventive Concept 100, wherein the electrical lead is bifurcated, so as to define a main portion and two or more bifurcation portions integral with respective bifurcation elongate portions of the elongate portion of the PCB.

Inventive Concept 102. The prosthetic cardiac valve according to Inventive Concept 100, wherein the electrical lead is one of a plurality of electrical leads, which are partially integral with the main elongate portion of the elongate portion of the PCB, and partially integral with respective bifurcation elongate portions of the elongate portion of the PCB.

Inventive Concept 103. The prosthetic cardiac valve according to any one of Inventive Concepts 72-102, wherein the prosthetic cardiac valve is a prosthetic aortic valve.

Inventive Concept 104. The prosthetic cardiac valve according to Inventive Concept 103, wherein the circuitry is mechanically coupled to the frame downstream of the prosthetic leaflets, and the electrode is mechanically coupled to the frame upstream of the prosthetic leaflets.

Inventive Concept 105. The prosthetic cardiac valve according to any one of Inventive Concepts 72-102, wherein the prosthetic cardiac valve is a prosthetic atrioventricular valve.

Inventive Concept 106. The prosthetic cardiac valve according to Inventive Concept 105, wherein the circuitry is mechanically coupled to the frame upstream of the prosthetic leaflets, and the electrode is mechanically coupled to the frame downstream of the prosthetic leaflets.

Inventive Concept 107. A prosthetic cardiac valve, which is configured to be delivered to a native cardiac valve of a patient in a constrained delivery configuration, and which comprises:
a frame, which defines a central longitudinal axis when the prosthetic cardiac valve is in the constrained delivery configuration, and which comprises interconnected stent struts arranged so as to define interconnected stent cells;
a plurality of prosthetic leaflets coupled to the frame so as to allow blood flow in a downstream direction and inhibit blood flow in an upstream direction when the prosthetic cardiac valve is in an expanded deployment configuration; and
an antenna, which is mechanically coupled to the frame, and which comprises:
   a magnetic core; and
   one or more coils, which are wound about the magnetic core,
wherein, at at least one location along a length of the magnetic core, a planar space bound by an outer perimeter of the magnetic core, in a plane perpendicular to the central longitudinal axis of the frame, has:
   (a) a shorter dimension, which is measured along a ray that (i) radiates radially outward from the central longitudinal axis and (ii) intersects a centroid defined by the planar space bound by the outer perimeter, and
   (b) a longer dimension, which is measured perpendicular to the shorter dimension in the plane, and is at least 150% of the shorter dimension.

Inventive Concept 108. The prosthetic cardiac valve according to Inventive Concept 107, wherein the longer dimension is at least 175% of the shorter dimension.

Inventive Concept 109. The prosthetic cardiac valve according to Inventive Concept 108, wherein the longer dimension is at least 200% of the shorter dimension.

Inventive Concept 110. The prosthetic cardiac valve according to Inventive Concept 108, wherein the longer dimension is no more than 400% of the shorter dimension.

Inventive Concept 111. The prosthetic cardiac valve according to Inventive Concept 107, wherein the longer dimension is no more than 350% of the shorter dimension.

Inventive Concept 112. The prosthetic cardiac valve according to Inventive Concept 107, wherein the central longitudinal axis intercepts the plane defined by outer perimeter outside the planar space.

Inventive Concept 113. The prosthetic cardiac valve according to Inventive Concept 107,
wherein an external surface of the magnetic core is shaped so as to define an axially-oriented groove,
wherein at least one of the one or more coils comprises a wire, and
wherein a straight portion of the wire is disposed at least partially within the axially-oriented groove, so as to pass from a first axial end to a second axial end of the at least one of the coils.

Inventive Concept 114. The prosthetic cardiac valve according to Inventive Concept 107, further comprising:
a cathode and an anode, which are mechanically coupled to the frame; and
circuitry, which is electrically coupled to the cathode, the anode, and the one or more coils.

Inventive Concept 115. The prosthetic cardiac valve according to any one of Inventive Concepts 107-114, wherein a radially-outward portion of the outer perimeter of the magnetic core, which includes a point on the outer perimeter farthest from the central longitudinal axis, is concavely curved with respect to the central longitudinal axis.

Inventive Concept 116. The prosthetic cardiac valve according to Inventive Concept 115, wherein the radially-outward portion of the outer perimeter of the magnetic core has a greatest radius of curvature of 1-5 mm.

Inventive Concept 117. The prosthetic cardiac valve according to Inventive Concept 115, wherein the radially-outward portion of the outer perimeter of the magnetic core has a greatest radius of curvature of 0.3-1.6 times the longer dimension.

Inventive Concept 118. The prosthetic cardiac valve according to Inventive Concept 115, wherein a radially-inward portion of the outer perimeter, which includes a point on the outer perimeter closest to the central longitudinal axis, is flat.

Inventive Concept 119. The prosthetic cardiac valve according to Inventive Concept 115, wherein a radially-inward portion of the outer perimeter, which includes one or more points on the outer perimeter closest to the central longitudinal axis, is concavely curved with respect to the central longitudinal axis.

Inventive Concept 120. The prosthetic cardiac valve according to Inventive Concept 119, wherein the radially-inward portion of the outer perimeter has a greatest radius of curvature that is less than a greatest radius of curvature of the radially-outward portion of the outer perimeter.

Inventive Concept 121. The prosthetic cardiac valve according to Inventive Concept 119, wherein the curved radially-outward portion of the outer perimeter includes an arcuate portion of a circle.

Inventive Concept 122. The prosthetic cardiac valve according to Inventive Concept 121, wherein the arcuate portion has a measure of 45-180 degrees.

Inventive Concept 123. The prosthetic cardiac valve according to Inventive Concept 122, wherein the measure is 60-120 degrees.

Inventive Concept 124. The prosthetic cardiac valve according to any one of Inventive Concepts 107-114,
wherein the magnetic core is shaped so as to define a cavity, and
wherein the prosthetic cardiac valve further comprises circuitry, which is disposed at least partially within the cavity, and which is electrically coupled to the one or more coils.

Inventive Concept 125. The prosthetic cardiac valve according to Inventive Concept 124, wherein the circuitry is disposed entirely within the cavity.

Inventive Concept 126. The prosthetic cardiac valve according to Inventive Concept 124, wherein the magnetic core has an average wall thickness surrounding the cavity of 100-500 microns.

Inventive Concept 127. The prosthetic cardiac valve according to Inventive Concept 124, wherein the magnetic core has an average wall thickness surrounding the cavity equal to 0.05-0.4 times the shorter dimension.

Inventive Concept 128. The prosthetic cardiac valve according to any one of Inventive Concepts 107-114,
wherein the magnetic core is elongate,
wherein the one or more coils comprise:
   first, second, and third coils, wound around the elongate magnetic core such that:
      the first coil encircles a first-coil longitudinal axis that coincides with a central longitudinal axis of the elongate magnetic core,
      the second coil encircles a second-coil longitudinal axis that is perpendicular to the first-coil longitudinal axis, and
      the third coil encircles a third-coil longitudinal axis that is perpendicular to the first-coil longitudinal axis and to the second-coil longitudinal axis,
wherein the second and the third coils cross each other at both longitudinal ends of the elongate magnetic core,
wherein the second coil has two longer sides and two shorter sides, and
wherein the two longer sides are parallel to the central longitudinal axis of the elongate magnetic core, or define an angle of less than 10 degrees with respect to the central longitudinal axis of the elongate magnetic core.

Inventive Concept 129. The prosthetic cardiac valve according to Inventive Concept 128,
wherein the third coil has two longer sides and two shorter sides, and
wherein the two longer sides of the third coil are parallel to the central longitudinal axis of the elongate magnetic core, or define an angle of less than 10 degrees with respect to the central longitudinal axis of the elongate magnetic core.

Inventive Concept 130. The prosthetic cardiac valve according to Inventive Concept 128, wherein the two longer sides cross the first coil at a plurality of first locations, and define angles of 75-90 degrees with the first coil at each of the plurality of first locations.

Inventive Concept 131. The prosthetic cardiac valve according to Inventive Concept 130,
wherein the third coil has two longer sides and two shorter sides, and
wherein the two longer sides of the third coil cross the first coil at a plurality of second locations, and define angles of 75-90 degrees with the first coil at each of the plurality of second locations.

Inventive Concept 132. The prosthetic cardiac valve according to Inventive Concept 128,
wherein an external surface of the elongate magnetic core is shaped so as to define an axially-oriented groove,
wherein the first coil comprises a wire, and
wherein a straight portion of the wire is disposed at least partially within the axially-oriented groove, so as to pass from a first axial end to a second axial end of the first coil.

Inventive Concept 133. The prosthetic cardiac valve according to any one of Inventive Concepts 107-132, wherein the frame further comprises one or more delivery-tool-coupling tabs, disposed proximal of the stent cells.

Inventive Concept 134. A valve prosthesis system comprising the prosthetic cardiac valve according to any one of Inventive Concepts 107-133, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

Inventive Concept 135. The prosthetic cardiac valve according to any one of Inventive Concepts 107-133, wherein the prosthetic cardiac valve is a prosthetic aortic valve, and wherein the antenna is mechanically coupled to the frame downstream of the prosthetic leaflets.

Inventive Concept 136. The prosthetic cardiac valve according to any one of Inventive Concepts 107-133, wherein the prosthetic cardiac valve is a prosthetic atrioventricular valve, and wherein the antenna is mechanically coupled to the frame upstream of the prosthetic leaflets.

Inventive Concept 137. The prosthetic cardiac valve according to any one of Inventive Concepts 107-133, wherein the antenna is mechanically coupled to the frame proximal of the prosthetic leaflets.

Inventive Concept 138. A valve prosthesis system comprising the prosthetic cardiac valve according to any one of Inventive Concepts 107-137, the valve prosthesis system further comprising an external unit,
wherein the one or more coils are one or more prosthetic-valve coils, and
wherein the external unit is configured to be disposed outside a body of the patient, and comprises:
   an energy-transmission coil; and
   external-unit control circuitry, which is configured to drive the energy-transmission coil to wirelessly transfer energy to at least one of the one or more prosthetic-valve coils by inductive coupling.

Inventive Concept 139. Apparatus comprising an implantable medical device, which comprises:
an antenna, which comprises:
   a magnetic core, which is shaped so as to define a cavity; and
   one or more coils, which are wound about the magnetic core; and
circuitry, which is disposed at least partially within the cavity, and which is electrically coupled to the one or more coils.

Inventive Concept 140. The apparatus according to Inventive Concept 139, wherein the circuitry is disposed entirely within the cavity.

Inventive Concept 141. The apparatus according to Inventive Concept 139, wherein the magnetic core has an average wall thickness surrounding the cavity of 100-500 microns.

Inventive Concept 142. The apparatus according to Inventive Concept 139, further comprising a cathode and an anode, which are electrically coupled to the circuitry.

Inventive Concept 143. The apparatus according to any one of Inventive Concepts 139-142, wherein the implantable medical device comprises a prosthetic cardiac valve, which is configured to be delivered to a native cardiac valve of a patient in a constrained delivery configuration, and which comprises:
a frame, which comprises interconnected stent struts arranged so as to define interconnected stent cells; and
a plurality of prosthetic leaflets coupled to the frame so as to allow blood flow in a downstream direction and inhibit blood flow in an upstream direction when the prosthetic cardiac valve is in an expanded deployment configuration,
wherein the antenna is mechanically coupled to the frame.

Inventive Concept 144. The apparatus according to Inventive Concept 143, wherein the antenna is mechanically coupled to the frame proximal of the prosthetic leaflets.

Inventive Concept 145. The apparatus according to Inventive Concept 143,
wherein the frame defines a central longitudinal axis when the prosthetic cardiac valve is in the constrained delivery configuration, and
wherein, at at least one location along a length of the magnetic core, a planar space bound by an outer perimeter of the magnetic core, in a plane perpendicular to the central longitudinal axis of the frame, has:
   (a) a shorter dimension, which is measured along a ray that (i) radiates radially outward from the central longitudinal axis and (ii) intersects a centroid defined by the planar space bound by the outer perimeter, and
   (b) a longer dimension, which is measured perpendicular to the shorter dimension in the plane, and is at least 150% of the shorter dimension.

Inventive Concept 146. A valve prosthesis system comprising the apparatus according to Inventive Concept 143, the valve prosthesis system further comprising an external unit,
wherein the one or more coils are one or more medical-device coils, and
wherein the external unit is configured to be disposed outside a body of the patient, and comprises:
   an energy-transmission coil; and
   external-unit control circuitry, which is configured to drive the energy-transmission coil to wirelessly transfer energy to at least one of the one or more medical-device coils by inductive coupling.

Inventive Concept 147. A prosthetic cardiac valve system comprising a prosthetic cardiac valve, which is configured to be delivered to a native cardiac valve of a heart of a patient in a constrained delivery configuration, and which comprises:
a frame;
a plurality of prosthetic leaflets coupled to the frame so as to allow blood flow in a downstream direction and inhibit blood flow in an upstream direction when the prosthetic cardiac valve is in an expanded deployment configuration;
electrodes, which include one or more cathodes and one or more anodes, which are mechanically coupled to the frame; and
circuitry, which is electrically coupled to the electrodes, and which is configured to apply pacing to the heart using a subset of the electrodes that includes fewer than all of the electrodes, at least one of the one or more cathodes, and at least one of the one or more anodes.

Inventive Concept 148. The prosthetic cardiac valve system according to Inventive Concept 147, wherein the prosthetic cardiac valve system is configured to select the subset of the electrodes by separately activating different combinations of the electrodes at different times, and selecting the subset of the electrodes that provides most effective pacing.

Inventive Concept 149. The prosthetic cardiac valve system according to Inventive Concept 148, wherein the prosthetic cardiac valve is configured to sense an ECG of the heart, and wherein the prosthetic cardiac valve system is configured to select the subset of the electrodes based on the ECG sensed when separately activating the different combinations of the electrodes at the different times.

Inventive Concept 150. The prosthetic cardiac valve system according to Inventive Concept 148, wherein the prosthetic cardiac valve system is configured to select the subset of the electrodes by separately activating different combinations of the electrodes before the circuitry applies each pulse of the pacing.

Inventive Concept 151. The prosthetic cardiac valve system according to Inventive Concept 148, wherein the circuitry of the prosthetic cardiac valve is configured to select the subset of the electrodes.

Inventive Concept 152. The prosthetic cardiac valve system according to Inventive Concept 148, wherein the circuitry is prosthetic-aortic-valve circuitry, and wherein the prosthetic cardiac valve system comprises an external control unit, which comprises external circuitry that is configured to select the subset of the electrodes.

Inventive Concept 153. The prosthetic cardiac valve system according to any one of Inventive Concepts 147-152, wherein the prosthetic cardiac valve is a prosthetic aortic valve.

Inventive Concept 154. The prosthetic cardiac valve system according to any one of Inventive Concepts 147-152, wherein the prosthetic cardiac valve is a prosthetic atrioventricular valve.

Inventive Concept 155. A valve prosthesis system for use with a guidewire, the valve prosthesis system comprising:
(i) a prosthetic cardiac valve, which is configured to be delivered to a native cardiac valve of a patient in a constrained delivery configuration using the guidewire, and which comprises:
   (a) a frame;
   (b) a plurality of prosthetic leaflets coupled to the frame;
   (c) a cathode and an anode, which are mechanically coupled to the frame; and
   (d) an antenna, which comprises one or more prosthetic-valve coils, and which is in electrical communication with the cathode and the anode; and
(ii) an external unit, which is configured to be disposed outside a body of the patient, and which comprises:
   (a) a housing, which is shaped so as to define a guidewire-receiving channel;
   (b) a rapid-pacing user control;
   (c) an energy-transmission coil; and
   (d) external-unit control circuitry, which is configured to:
      drive the energy-transmission coil to wirelessly transfer energy to at least one of the one or more prosthetic-valve coils by inductive coupling, and
      only upon activation of the rapid-pacing user control and when the guidewire is disposed within the guidewire-receiving channel of the housing, drive the prosthetic cardiac valve to apply rapid pacing using the cathode and the anode.

Inventive Concept 156. The valve prosthesis system according to Inventive Concept 155, wherein the prosthetic cardiac valve is a prosthetic aortic valve.

Inventive Concept 157. The valve prosthesis system according to Inventive Concept 155, wherein the prosthetic cardiac valve is a prosthetic atrioventricular valve.

Inventive Concept 158. Apparatus comprising an implantable medical device, which comprises:
an antenna, which comprises:
   an elongate core; and
   first, second, and third coils, wound around the elongate core such that:
      the first coil encircles a first-coil longitudinal axis that coincides with a central longitudinal axis of the elongate core,
      the second coil encircles a second-coil longitudinal axis that is perpendicular to the first-coil longitudinal axis, and
      the third coil encircles a third-coil longitudinal axis that is perpendicular to the first-coil longitudinal axis and to the second-coil longitudinal axis,
wherein the second and the third coils cross each other at both longitudinal ends of the elongate core,
wherein the second coil has two longer sides and two shorter sides, and
wherein the two longer sides are parallel to the central longitudinal axis of the elongate core, or define an angle of less than 10 degrees with respect to the central longitudinal axis of the elongate core.

Inventive Concept 159. The apparatus according to Inventive Concept 158,
wherein the third coil has two longer sides and two shorter sides, and
wherein the two longer sides of the third coil are parallel to the central longitudinal axis of the elongate core, or define an angle of less than 10 degrees with respect to the central longitudinal axis of the elongate core.

Inventive Concept 160. The apparatus according to Inventive Concept 158, wherein the two longer sides cross the first coil at a plurality of first locations, and define angles of 75-90 degrees with the first coil at each of the plurality of first locations.

Inventive Concept 161. The apparatus according to Inventive Concept 160,
wherein the third coil has two longer sides and two shorter sides, and
wherein the two longer sides of the third coil cross the first coil at a plurality of second locations, and define angles of 75-90 degrees with the first coil at each of the plurality of second locations.

Inventive Concept 162. The apparatus according to Inventive Concept 158,
wherein an external surface of the elongate core is shaped so as to define an axially-oriented groove,
wherein the first coil comprises a wire, and
wherein a straight portion of the wire is disposed at least partially within the axially-oriented groove, so as to pass from a first axial end to a second axial end of the first coil.

Inventive Concept 163. A prosthetic cardiac valve system comprising a prosthetic cardiac valve, which is configured to be delivered to a native cardiac valve of a heart of a patient in a constrained delivery configuration, and which comprises:
a frame, which comprises interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to apply pacing to the heart by activating one or more of the distal electrodes as one or more anodes and one or more of the other distal electrodes as one or more cathodes.

Inventive Concept 164. The prosthetic cardiac valve system according to Inventive Concept 163, wherein the distal electrodes are mechanically coupled to the frame at or within 8 mm of the respective ones of the distal peaks.

Inventive Concept 165. The prosthetic cardiac valve system according to Inventive Concept 163, wherein the distal stent cells are distal-most ones of the stent cells.

Inventive Concept 166. The prosthetic cardiac valve system according to Inventive Concept 163, wherein the distal peaks are respective upstream peaks, and wherein the distal electrodes are upstream electrodes that are mechanically coupled to the frame at or near respective ones of the upstream peaks.

Inventive Concept 167. The prosthetic cardiac valve system according to any one of Inventive Concepts 163-166, wherein the prosthetic cardiac valve is a prosthetic aortic valve.

Inventive Concept 168. The prosthetic cardiac valve system according to any one of Inventive Concepts 163-166, wherein the prosthetic cardiac valve is a prosthetic atrioventricular valve.

The present invention will be more fully understood from the following detailed description of embodiments thereof, taken together with the drawings, in which:

### BRIEF DESCRIPTION OF THE DRAWINGS

FIGS. 1A and 1B are schematic illustrations a prosthetic aortic valve, in accordance with an application of the present invention;

FIG. 2 is a schematic illustration of a valve prosthesis system and the prosthetic aortic valve of FIGS. 1A-B implanted in a body of a patient, in accordance with an application of the present invention;

FIGS. 3A-C are schematic illustrations of a printed circuit board (PCB), an electrical lead, and electrodes, in accordance with an application of the present invention;

FIG. 3D is a schematic illustration of a portion of the prosthetic aortic valve of FIGS. 1A-B and the PCB of FIGS. 3A-C coupled to stent struts of the prosthetic aortic valve, in accordance with an application of the present invention;

FIGS. 3E and 3F are schematic illustrations additional configurations of the PCB of FIGS. 3A-C, in accordance with respective applications of the present invention;

FIG. 3G is a schematic illustration of another configuration of a frame of the prosthetic aortic valve of FIGS. 1A-B and the PCB of FIGS. 3A-C coupled to the frame, in accordance with an application of the present invention;

FIG. 3H is a schematic illustration of yet another configuration of the PCB of FIGS. 3A-C, in accordance with an application of the present invention;

FIG. 4 is a schematic illustration of a portion of the prosthetic aortic valve of FIGS. 1A-B in a constrained delivery configuration within a delivery sheath, in accordance with an application of the present invention;

FIGS. 5A-B are schematic illustrations of a magnetic core of an antenna, in accordance with an application of the present invention;

FIGS. 6A-B are schematic illustrations of another configuration of the magnetic core of the antenna of FIGS. 5A-B, in accordance with an application of the present invention;

FIG. 7 is a schematic illustration of yet another configuration of the magnetic core of the antenna of FIGS. 5A-B, in accordance with an application of the present invention;

FIGS. 8A and 8B are schematic illustrations of another antenna, in accordance with respective applications of the present invention;

FIG. 9 is a schematic illustration of a prosthetic atrioventricular valve, in accordance with an application of the present invention;

FIG. 10 is a schematic illustration of a frame of the prosthetic aortic valve of FIGS. 1A-B when the prosthetic aortic valve is in an expanded deployment configuration, in accordance with an application of the present invention; and

FIG. 11 is a schematic illustration of an external control unit of a valve prosthesis system comprising the prosthetic aortic valve of FIGS. 1A-B, in accordance with an application of the present invention.

### DETAILED DESCRIPTION OF APPLICATIONS

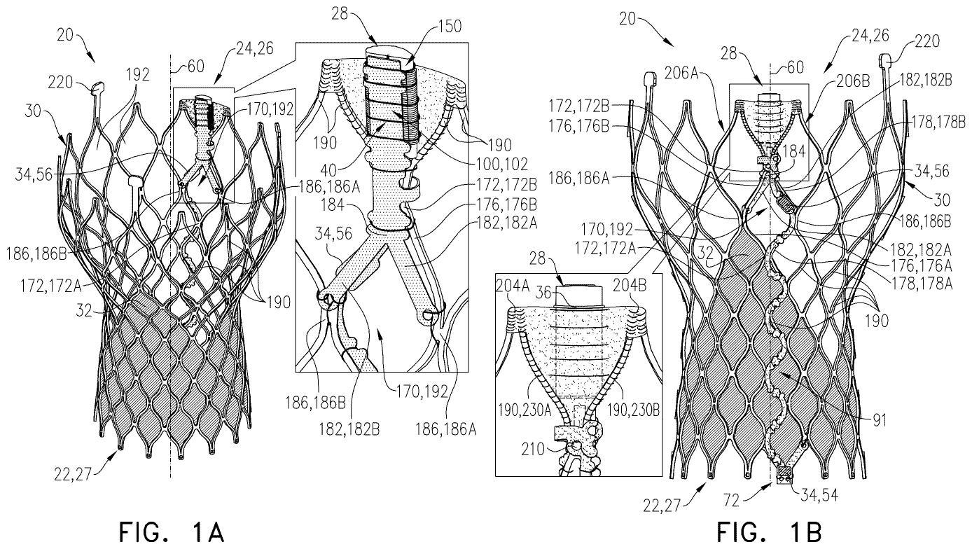

Reference is made to FIGS. 1A and 1B, which are schematic illustrations of a prosthetic aortic valve 20, in accordance with an application of the present invention. For clarity of illustration, only the closer half of prosthetic aortic valve 20 is shown in FIG. 1B.

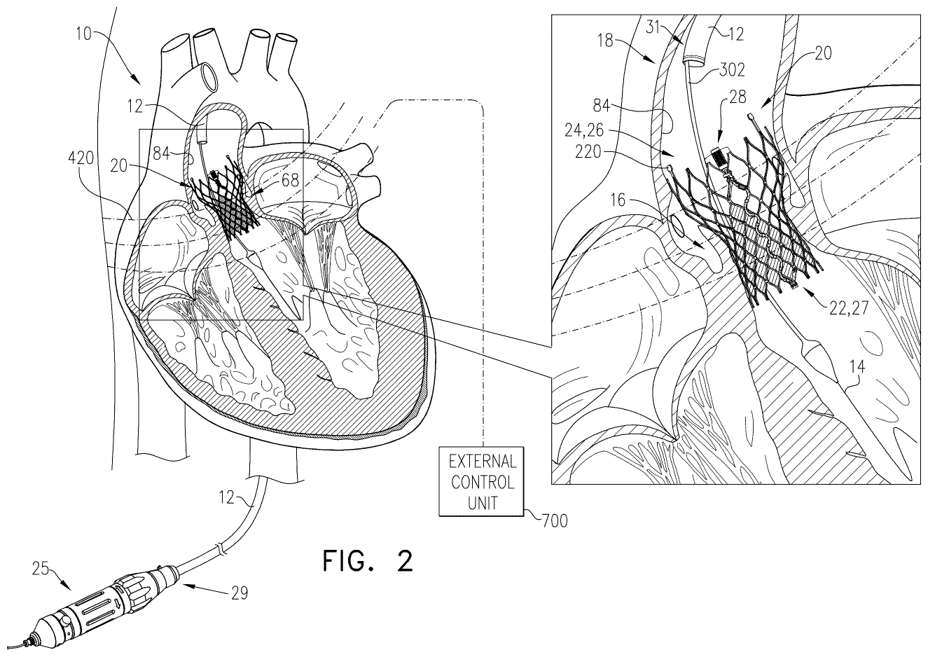

Reference is also made to FIG. 2, which is a schematic illustration of a valve prosthesis system 10 and prosthetic aortic valve 20 implanted in a body of a patient, in accordance with an application of the present invention. Valve prosthesis system 10 further comprises a delivery system 18, which typically comprises a delivery sheath 12 and is used with a guidewire 14. Delivery system 18 typically further comprises a user-control handle 25, which is disposed at (and optionally coupled to) a proximal end portion 29 of delivery sheath 12. The opposite, free end portion of delivery sheath 12 is thus a distal end portion 31 of delivery sheath 12. Prosthetic aortic valve 20 is typically configured to be delivered to a native aortic valve 16 of the patient in a constrained delivery configuration within delivery sheath 12. Distal end portion 31 of delivery sheath 12 may be a conventional tube, for example as shown. Alternatively, distal end portion 31 of delivery sheath 12 may further comprise a capsule that is moveable distally with respect to the remainder of delivery sheath 12 during deployment. All or a portion of prosthetic aortic valve 20 may be contained within the capsule. As used in the present application, including in the claims and Inventive Concepts, in configurations in which distal end portion 31 comprises a capsule (or other type of holder), the distal end portion 31 of delivery sheath 12 refers to the combination of the conventional tubular portion of the sheath and the capsule. By way of example and not limitation, such a capsule is described in U.S. Pat. No. 10,888,421 to Hariton et al., which is incorporated herein by reference.

Typically, prosthetic aortic valve 20 is deployed using imaging, such as fluoroscopy, and is rotated if necessary during the deployment such that a cathode 54 is disposed against tissue of the annulus that is near the bundle of His.

Prosthetic aortic valve 20 is shown in FIGS. 1A-B and FIG. 2 in an expanded configuration. Frame 30 defines a central longitudinal axis 60 when prosthetic aortic valve 20 is in this expanded deployment configuration. Prosthetic aortic valve 20 has an upstream end 22 and a downstream end 24. Downstream end 24 may also be a proximal end 26 and upstream end 22 may also be a distal end 27, for example because proximal end 26 may be disposed in distal end portion 31 of delivery sheath 12 more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic aortic valve 20, to delivery system 18, e.g., to a delivery shaft of delivery system 18, such as described hereinbelow). For other applications (configuration not shown), distal end 27 is configured to be coupled to delivery system 18, such as to a capsule of the distal end portion 31 of delivery sheath 12, such as described hereinabove.

Prosthetic Aortic Valve 20 Comprises:
a Frame 30;
a plurality of prosthetic leaflets 32 coupled to frame 30 so as to allow blood flow in a downstream direction and inhibit blood flow in an upstream direction when prosthetic aortic valve 20 is in the expanded deployment configuration, such as shown in FIGS. 1A-B and 2;
an antenna 28, which is mechanically coupled to frame 30, and which comprises one or more prosthetic-valve coils 36;
one or more electrodes 34, such as cathode 54 and an anode 56, coupled to frame 30; and
optionally, circuitry 40, which is electrically coupled to cathode 54, anode 56, and the one or more prosthetic-valve coils 36.

Typically, circuitry 40 is configured to apply pacing to the heart using the one or more electrodes 34. For example, the pacing may be applied temporarily for up to several weeks after implantation of prosthetic aortic valve 20 (e.g., up to one month after implantation), typically using an external control unit to continuously provide power, such as external control unit 400, described hereinbelow with reference to FIG. 11.

Alternatively, for some applications, the pacing is applied longer-term, in which case valve prosthesis system 10 may comprise an energy storage module, e.g., comprising a battery. For example, prosthetic aortic valve 20 may further comprise the energy storage module, e.g., comprising a battery, which may be periodically charged using the external control unit, which may obviate the need for the patient to constantly wear an external energy transmitter. Alternatively or additionally, for example, valve prosthesis system 10 may comprise an implantable energy storage module, e.g., comprising a battery (e.g., a rechargeable battery); for example, the energy storage module may be implantable subcutaneously. The implantable energy storage unit may provide power to prosthetic aortic valve 20 either wirelessly and/or wiredly. For example, the pacing may comprise ongoing sensing of a native electrical signal of the heart and deliverance of electrical stimulus in cases in which the native signal is unsatisfactory for timely ventricular contraction (“VVI pacing”).

Further alternatively or additionally, for some applications, circuitry 40 is configured to apply rapid pacing during an invasive structural heart procedure, such as an implantation procedure, such as a TAVR-in-TAVR procedure in which the first TAVR comprises prosthetic aortic valve 20.

For some applications, prosthetic aortic valve 20 is configured to sense an electrocardiography (ECG) of the patient's heart. Circuitry 40 may be configured to sense the ECG, or separate circuitry may be provided for sensing the ECG. The ECG sensing may be performed using all or a subset of electrodes 34 and/or one or more separate electrodes may be provided for performing the ECG sensing.

Each of the one or more prosthetic-valve coils 36 comprises an electrically conductive wire coated with electrical insulation.

Frame 30 typically comprises a stent or other structure, which is typically self-expanding, and may be formed by laser cutting or etching a metal alloy tube comprising, for example, stainless steel or a shape memory material such as Nitinol. For some applications, frame 30 comprises interconnected stent struts 190 arranged so as to define interconnected stent cells 192. Optionally, interconnected stent cells 192 are generally diamond-shaped, such as shown in the drawings.

Typically, adjoining pairs of prosthetic leaflets 32 are attached to one another at their lateral ends to form commissures, with free edges of the prosthetic leaflets forming coaptation edges that meet one another. Prosthetic leaflets 32 typically comprise a sheet of animal pericardial tissue, such as porcine pericardial tissue, or synthetic or polymeric material. Optionally, prosthetic aortic valve 20 further comprises a skirt.

For some applications, cathode 54 has a thickness of at least 10 microns, no more than 200 microns, and/or between 10 and 200 microns, e.g., about 50 microns, and/or a surface area of at least 0.5 mm\hat{ }2, e.g., at least 1 mm\hat{ }2; no more than 20 mm\hat{ }2; and/or 0.5-20 mm\hat{ }2, such as 1-20 mm\hat{ }2, in order to provide adequate stimulation. For some applications, cathode 54 is coated with titanium nitride (TiN).

Typically, antenna 28 is mechanically coupled to frame 30 proximal (e.g., downstream) of prosthetic leaflets 32, such as shown. Alternatively, antenna 28 is mechanically coupled to frame 30 distal of prosthetic leaflets 32, or at least partially axially overlapping with prosthetic leaflets 32 (configurations not shown).

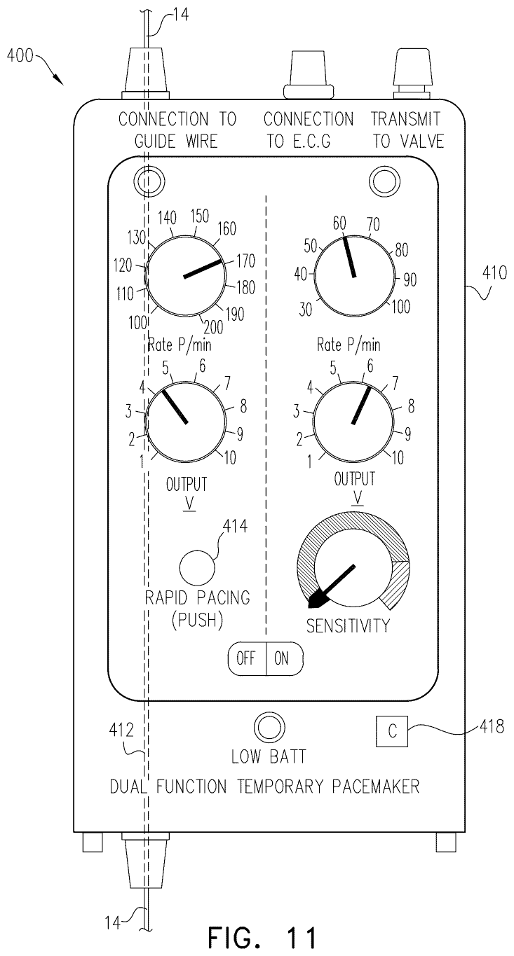

Reference is made to FIGS. 1A-B. For some applications:
circumferentially adjacent first and second proximal (e.g., downstream)-most stent cells 206A and 206B of interconnected stent cells 192 are joined at a cell junction 210 (labeled in FIG. 1B and better seen in FIG. 10, described hereinbelow),
first proximal (e.g., downstream)-most stent cell 206A comprises a right proximal (e.g., downstream) strut 230A of interconnected stent struts 190, right proximal (e.g., downstream) strut 230A extending between cell junction 210 and a first proximal (e.g., downstream) peak 204A defined by first proximal (e.g., downstream)-most stent cell 206A, and
second proximal (e.g., downstream)-most stent cell 206B comprises a left proximal (e.g., downstream) strut 230B of interconnected stent struts 190, left proximal (e.g., downstream) strut 230B extending between cell junction 210 and a second proximal (e.g., downstream) peak 204B defined by second proximal (e.g., downstream)-most stent cell 206B.

For some applications, prosthetic aortic valve 20 further comprises a flexible sheet 62, which is mechanically coupled to right and left proximal (e.g., downstream) struts 230A and 230B. Optionally, flexible sheet 62 is mechanically coupled to right and left proximal (e.g., downstream) struts 230A and 230B by stitching, such as shown; alternatively or additionally, flexible sheet 62 is mechanically coupled to right and left proximal (e.g., downstream) struts 230A and 230B using alternative coupling techniques that are known in the art.

Flexible sheet 62 may comprise, for example, a polymer (e.g., polyethylene terephthalate (PET) or expanded Polytetrafluoroethylene (ePTFE)) or biological tissue, e.g., a pericardium sheet. Optionally, the material of flexible sheet 62 is woven. Optionally, the material of flexible sheet 62 comprises cloth. Flexible sheet 62 is collapsible with prosthetic aortic valve 20 when loaded into delivery sheath 12.

Antenna 28 is mechanically coupled to frame 30 at least in part by being mechanically coupled to flexible sheet 62 between right and left proximal (e.g., downstream) struts 230A and 230B. Optionally, antenna 28 is mechanically coupled to flexible sheet 62 by stitching, such as shown; alternatively or additionally, antenna 28 is mechanically coupled to flexible sheet 62 using alternative coupling techniques that are known in the art. (Because flexible sheet 62 and antenna 28 are shown from outside prosthetic aortic valve 20 in FIG. 1B, flexible sheet 62 partially obscures the view of antenna 28.)

Optionally, antenna 28 is mechanically coupled to frame 30 at least in part by being mechanically coupled to cell junction 210.

For some applications, flexible sheet 62 has an area of 25-100 mm\hat{ }2.

For some applications, flexible sheet 62 is coupled only to one or more interconnected stent struts 190 of each of first and second proximal (e.g., downstream)—most stent cells 206A and 206B, and not to any interconnected stent struts 190 of other stent cells of frame 30.

For some applications, flexible sheet 62 has three sides.

Typically, flexible sheet 62 is separate and distinct from material of prosthetic leaflets 32.

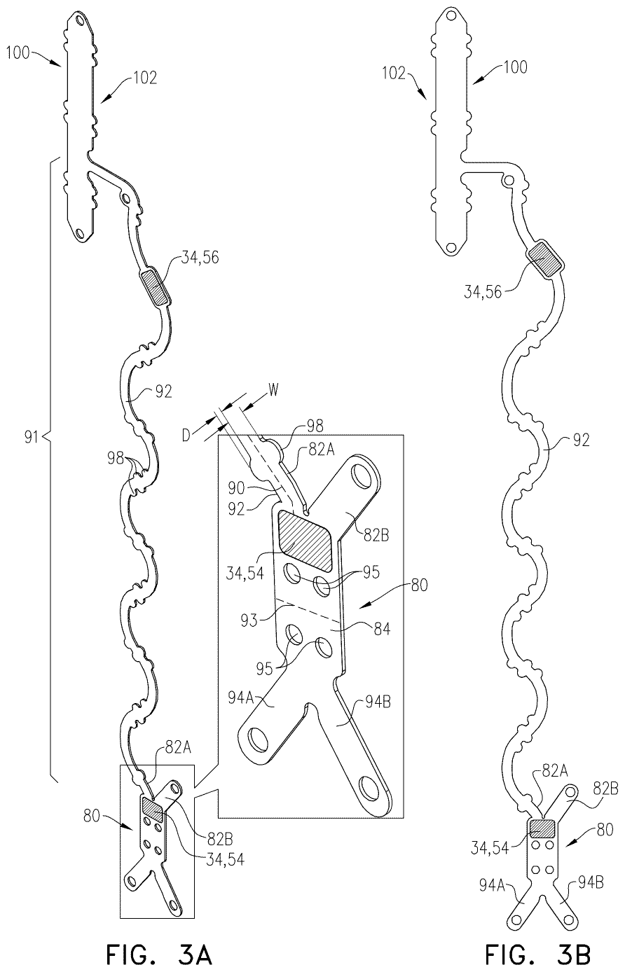

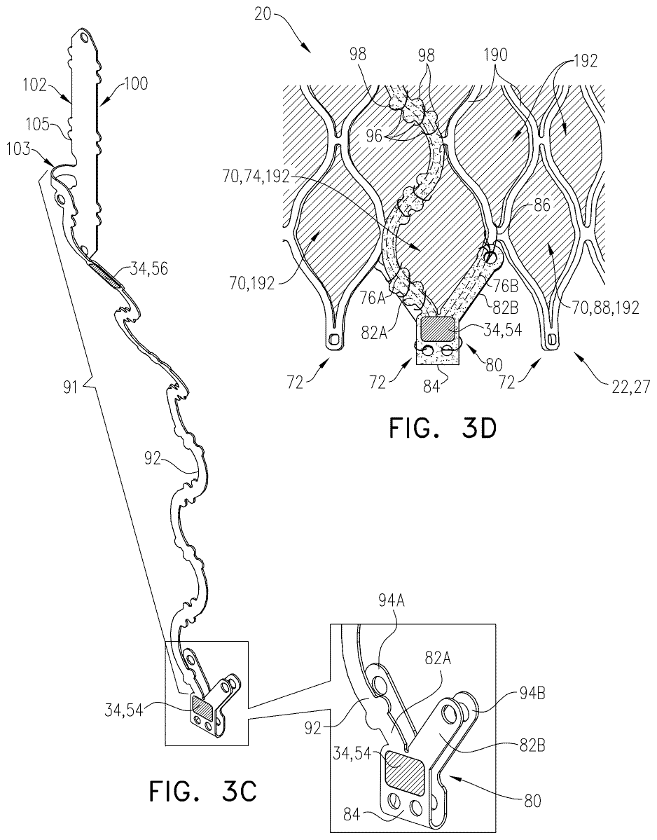

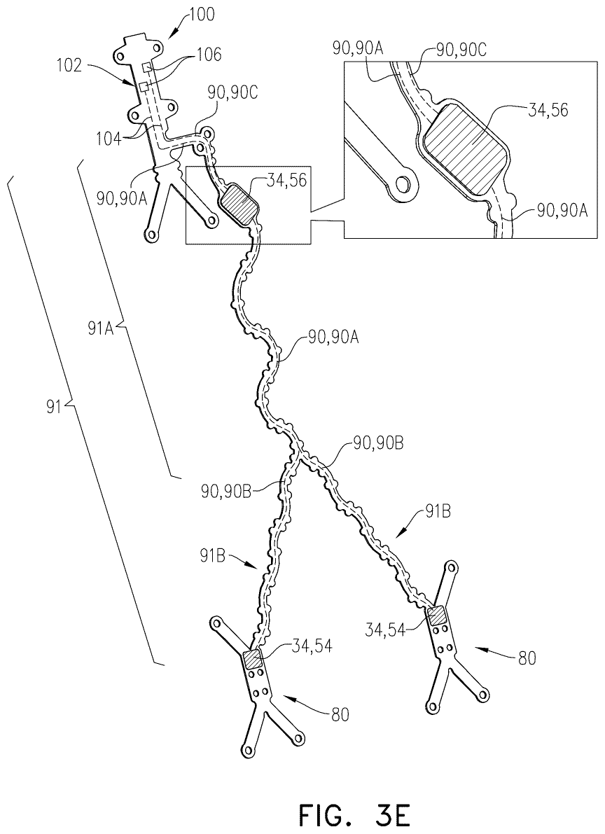

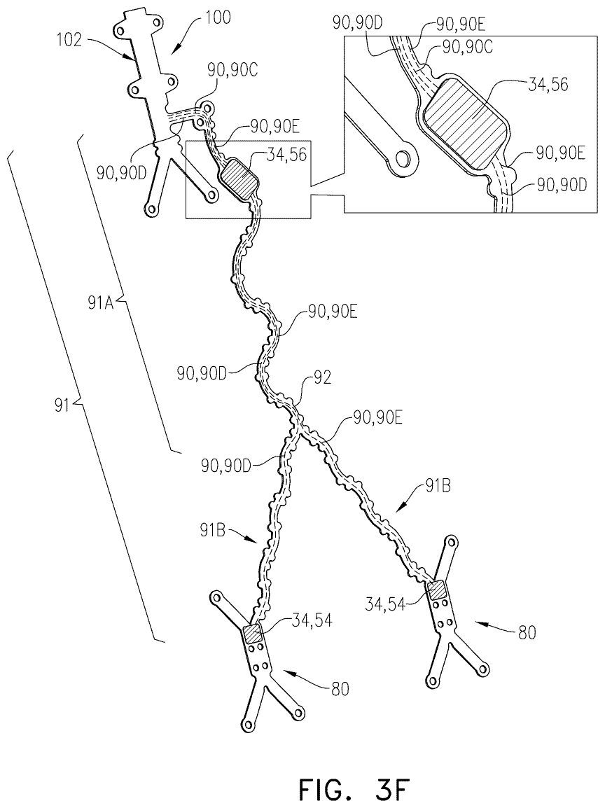

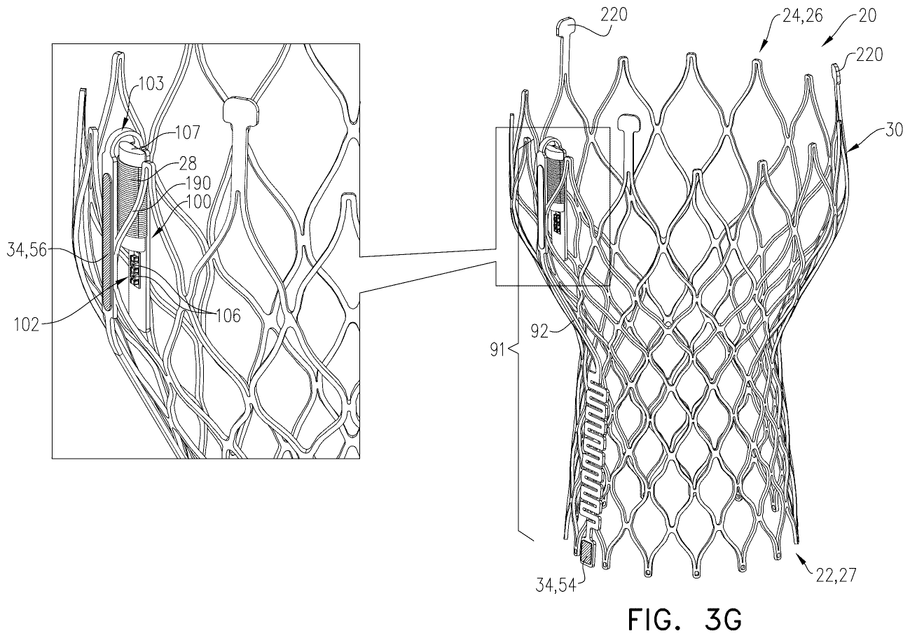

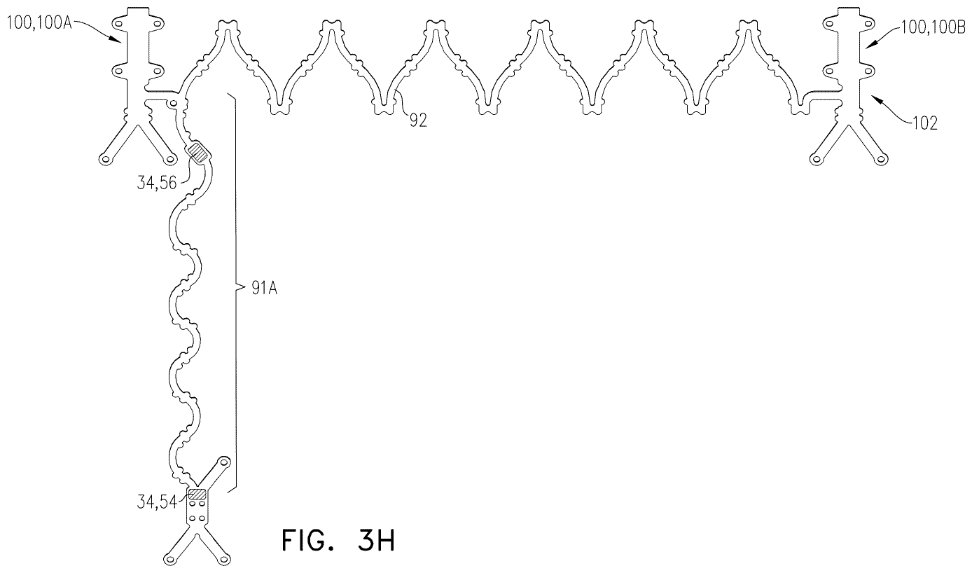

Reference is now made to FIGS. 3A-C, which are schematic illustrations of a printed circuit board (PCB) 92, an electrical lead 90, and electrodes 34, in accordance with an application of the present invention. PCB 92 typically comprises a polymer, such as polyimide, as is known the PCB art. PCB 92 is typically flexible.

Reference is also made to FIG. 3D, which is a schematic illustration of a portion of prosthetic aortic valve 20 and PCB 92 coupled to stent struts 190, in accordance with an application of the present invention.

Reference is further made to FIGS. 3E and 3F, which are schematic illustrations additional configurations of PCB 92, in accordance with respective applications of the present invention.

Reference is still further made to FIG. 3G, which is a schematic illustration of frame 30 of prosthetic aortic valve 20 and another configuration of PCB 92 coupled to frame 30, in accordance with an application of the present invention.

Reference is additionally made to FIG. 3H, which is a schematic illustration of yet another configuration of PCB 92, in accordance with an application of the present invention.

FIGS. 3A-C, 3E-F, and 3H show elements of prosthetic aortic valve 20 prior to assembly of prosthetic aortic valve 20, and FIGS. 3D and 3G show these elements after assembly of prosthetic aortic valve 20. For clarity of illustration, FIG. 3G does not show leaflets 32, although they provided in practice.

In some of the configurations shown in FIGS. 3A-F and 3H (and FIGS. 1A-B), distal (e.g., upstream) ones 70 of interconnected stent cells 192 are located in a distal (e.g., upstream) half of frame 30 and define respective distal (e.g., upstream) peaks 72. At least one electrode 34, such as a cathode 54 (as labeled) or an anode 56 (configuration not labeled), is disposed at or near (e.g., within 8 mm of) a distal (e.g., upstream) peak 72 of one 74 of the distal (e.g., upstream) stent cells 70 (and is thus referred to herein as a distal (e.g., upstream) electrode 34). First and second distal (e.g., upstream) stent struts 76A and 76B of the one 74 of distal (e.g., upstream) stent cells 70 are joined at the distal (e.g., upstream) peak 72 (the distal (e.g., upstream) peak 72 is obscured in FIG. 3D, but can be seen in the adjacent stent cells). Optionally, such as shown, the distal (e.g., upstream) ones 70 of stent cells 192 are distal (e.g., upstream)-most ones of stent cells 192, and the one 74 of distal (e.g., upstream) stent cells 70 is one 74 of distal (e.g., upstream)-most stent cells 192. Alternatively, the distal (e.g., upstream) ones 70 of stent cells 192 are not distal (e.g., upstream)-most ones of stent cells 192, and the one 74 of distal (e.g., upstream) stent cells 70 is not one 74 of distal (e.g., upstream)—most stent cells 192 (configuration not shown).

In some of the configurations shown in FIGS. 3A-F and 3H (and FIGS. 1A-B), at least one electrode 34, such as an anode 56 (as labeled) or a cathode 54 (configuration not labeled), is disposed on a proximal (e.g., downstream) portion of frame 30, such as (a) a proximal (e.g., downstream) half of frame 30, (b) a portion of frame 30 proximal (e.g., downstream) of prosthetic leaflets 32, and/or (c) a portion of frame defined by a proximal-most (e.g., downstream-most) two rows of stent cells. This at least one electrode 34 is thus referred to herein as a proximal (e.g., downstream) electrode 34.

In some of the configurations shown in FIGS. 3A-F and 3H, prosthetic aortic valve 20 further comprises coupling material 80, which is shaped so as to define:
a first strip 82A that is mechanically coupled to first distal (e.g., upstream) stent strut 76A,
a second strip 82B that is mechanically coupled to second distal (e.g., upstream) stent strut 76B, and
a junction 84, which couples together first and second strips 82A and 82B,
   such that first and second strips 82A and 82B together couple electrode 34, such as a cathode 54, to frame 30 at or near (e.g., within 8 mm of) distal (e.g., upstream) peak 72. Using first and second strips 82A and 82B in this arrangement to couple electrode 34 to frame 30 typically helps stabilize electrode 34 with respect to frame 30, both during expansion of frame 30 from its compressed elongated state, and during many cardiac cycles after implantation of frame 30.

Optionally, first and second strips 82A and 82B are integrally joined at junction 84, e.g., integrally formed from a single piece of material (such as shown); alternatively, first and second strips 82A and 82B comprise discrete pieces of material coupled together at junction 84 (configuration not shown). First strip 82A may be mechanically coupled to either surface of first distal (e.g., upstream) stent strut 76A, and second strip 82B may be mechanically coupled to either surface of second distal (e.g., upstream) stent strut 76B.

For some applications, first and second strips 82A and 82B are mechanically coupled to first and second distal (e.g., upstream) stent struts 76A and 76B, respectively, by stitching, such as shown (to this end, first and second strips 82A and 82B may comprise stitching holes, as shown).

For some applications, junction 84 of coupling material 80 is mechanically coupled to frame 30 at or near (e.g., within 5 mm of) distal (e.g., upstream) peak 72.

For some applications:
first strip 82A has length equal to at least 50% of a length of first distal (e.g., upstream) stent strut 76A; for example, the length of first strip 82A may be greater than the length of first distal (e.g., upstream) stent strut 76A, such as at least 120% of the length of first distal (e.g., upstream) stent strut 76A (which may aid with mechanically coupling first strip 82A to first distal (e.g., upstream) stent strut 76A), and/or
second strip 82B has length equal to at least 50% of a length of second distal (e.g., upstream) stent strut 76B, such as least 75%, e.g., 100% of the length of second distal (e.g., upstream) stent strut 76B, and/or no more than 100% of the length of second distal (e.g., upstream) stent strut 76B.

For some applications, the one 74 of distal (e.g., upstream) stent cells 70 is a first one 74 of distal (e.g., upstream) stent cells 70, and the first one 74 of distal (e.g., upstream) stent cells 70 is joined at a cell junction 86 (node) to a circumferentially-adjacent second one 88 of distal (e.g., upstream) stent cells 70. Second strip 82B is mechanically coupled to cell junction 86, such as by stitching, such as shown (to this end, second strip 82B may comprise a stitching hole, as shown).

For some applications, prosthetic aortic valve 20 further comprises electrical lead 90 (shown schematically in the enlargement in FIG. 3A), which is electrically coupled to electrode 34 (and typically circuitry 40, if provided). First strip 82A is mechanically coupled to at least a portion of electrical lead 90.

For some of these applications, first strip 82A comprises electrical insulation, and first strip 82A electrically insulates the at least a portion of electrical lead 90 (such that first strip 82A and electrical lead 90 together provide an electrode lead). For some of these applications, first strip 82A comprises an elongate portion 91 of PCB 92 with which electrical lead 90 is integral (e.g., encased within PCB 92, such as by lamination, or disposed on an external surface of PCB 92 and coated with an electrically insulating coating). Typically, electrical lead 90 comprises a track (also known as a conductive trace) of PCB 92. In this configuration, PCB 92 typically also defines second strip 82B and junction 84 of coupling material 80. Although elongate portion 91 of PCB 92 is shown as oriented in a generally distal-proximal (e.g., upstream-downstream) orientation, elongate portion 91 of PCB 92 may also be at least partially oriented in a circumferential (angular) orientation around a portion of frame 30, such as shown in FIG. 3H, in which PCB 92 is shaped so as to define a generally distal-proximal (e.g., upstream-downstream) oriented elongate portion labeled 91, as well as a circumferentially-oriented (angularly-oriented) elongate portion oriented circumferentially (angularly) around a circumferential (angular) portion of frame 30 (horizontal in the figure).

Alternatively, first strip 82A is non-electrically-insulating, in which case electrical lead 90 may be electrically insulated by separate electrical insulation.

For some applications, first and second strips 82A and 82B are outer first and second strips 82A and 82B, which are mechanically coupled to radially outer (with respect to central longitudinal axis 60 of frame 30) sides of first and second distal (e.g., upstream) stent struts 76A and 76B, respectively. Coupling material 80 is shaped so as to further define:
an inner first strip 94A that is mechanically coupled to a radially inner side of first distal (e.g., upstream) stent strut 76A, and
an inner second strip 94B that is mechanically coupled to a radially inner side of second distal (e.g., upstream) stent strut 76B.

Junction 84 of coupling material 80 couples together outer first strip 82A, outer second strip 82B, inner first strip 94A, and inner second strip 94B. Outer first strip 82A, outer second strip 82B, inner first strip 94A, and inner second strip 94B together couple electrode 34 to frame 30 at or near distal (e.g., upstream) peak 72.

For some of these applications, junction 84 of coupling material 80 is folded over distal (e.g., upstream) peak 72, such as shown, such as shown in FIGS. 3C-D. Optionally, the folded junction 84 is mechanically coupled to frame 30 at or near distal (e.g., upstream) peak 72, such as by stitching, such as shown (to this end, junction 84 may comprise stitching holes 95, as shown). Prior to being folded over during assembly of prosthetic aortic valve 20, junction 84 may generally have an X-shape, such as shown in FIGS. 3A-B. A fold line 93 is schematically labeled in the enlargement of FIG. 3A.

Reference is still made to FIGS. 3A-D, and is again made to FIGS. 1A-B. For some applications, electrical lead 90, which electrically couples one or more electrodes 34 to circuitry 40, is integral with elongate portion 91 of PCB 92 (electrical lead 90 is shown schematically in the enlargement of FIG. 3A). As shown in FIGS. 1A-B and 3D, elongate portion 91 of PCB 92 is mechanically coupled to some of interconnected stent struts 190 of frame 30, such as by suturing using sutures 96. Elongate portion 91 of PCB 92 thus serves both to provide electrical insulation to electrical lead 90 and to facilitate coupling of electrical lead 90 to stent struts 190. This encasing of electrical lead 90 in elongate portion 91 of PCB 92 may be implemented either in combination with the techniques for mechanically coupling junction 84 to frame 30 at or near distal (e.g., upstream) peak 72 described above with reference to FIGS. 3A-D, or independently of these techniques.

As described above, for some applications, which electrical lead 90, which electrically couples one or more electrodes 34 to circuitry 40, is integral with elongate portion 91 of PCB 92. For some of these applications, the one or more electrodes 34 are mechanically coupled to frame 30:
downstream of prosthetic leaflets 32 and/or at a proximal (e.g., downstream) half of frame 30, such as shown for anode 56 in FIGS. 1A-B, and/or
upstream of the prosthetic leaflets 32, optionally at or near (e.g., within 8 mm of) respective distal (e.g., upstream) peaks 72 of respective ones 74 of distal (e.g., upstream)-most ones 70 of stent cells 192.
   In any of these configurations, the one or more electrodes 34 may be directly coupled to frame 30, or may be indirectly coupled to frame 30 by being coupled to elongate portion 91 of PCB 92, which in turn is directly coupled to frame 30. In addition, in any of these configurations, circuitry 40 may be mechanically coupled to frame 30 proximal (e.g., downstream) of prosthetic leaflets 32.

For some applications, elongate portion 91 of PCB 92 has an undulating shape that generally runs along interconnected stent struts 190, such as shown in FIGS. 1A-B, 3D.

Alternatively or additionally, for some applications, elongate portion 91 of PCB is shaped so as to follow a path of interconnected stent struts 190, such as shown in FIGS. 1A-B and 3D, and/or has a same general shape as interconnected stent struts 190, also as shown in FIGS. 1A-B and 3D.

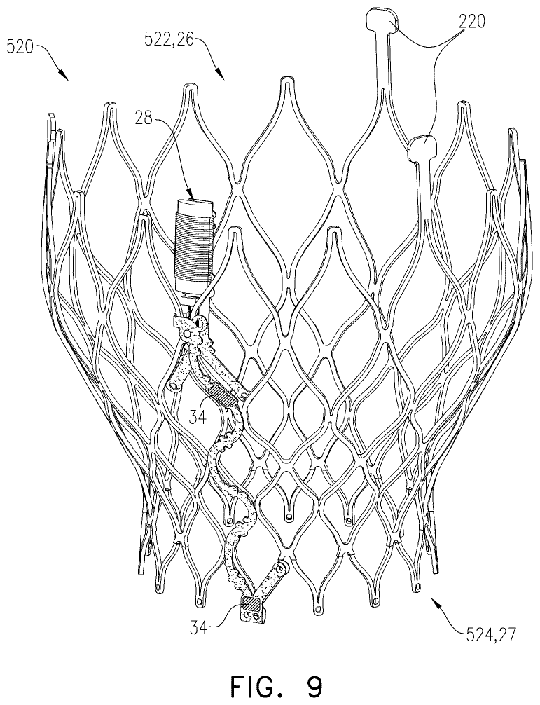

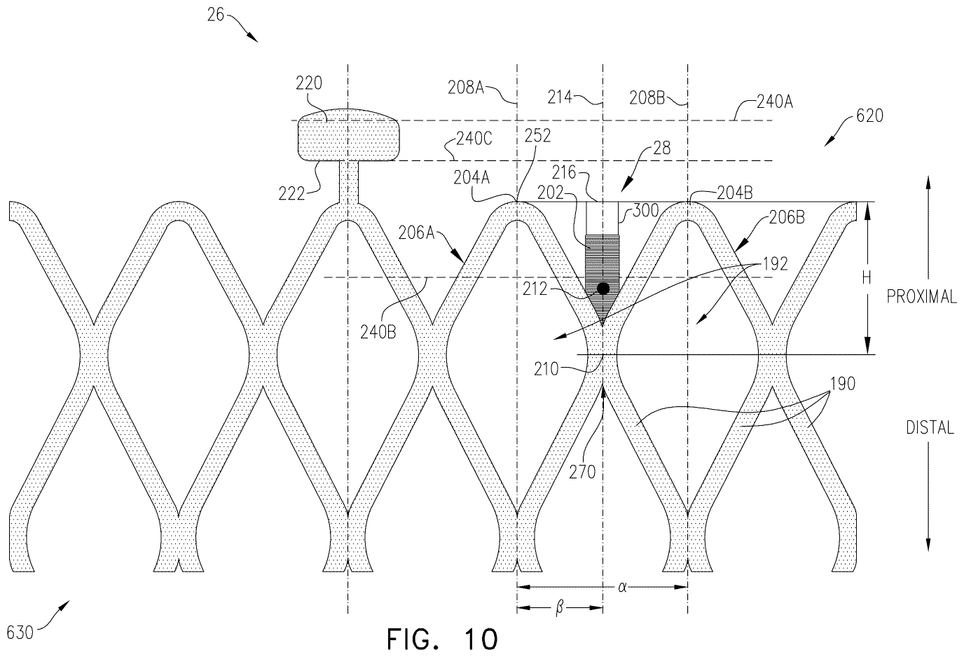

For some applications, elongate portion 91 of PCB 92 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92 (in configurations in which circuitry portion 100 is elongate, the greatest dimension may equal the length of long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for prosthetic aortic valve 20), or at least 1.5 cm, no more than 6 cm, and/or 1.5-6 cm (e.g., for the mitral or tricuspid valve, such as described hereinbelow with reference to FIG. 9).
   All of the above-mentioned lengths of elongate portion 91 are measured in a straight line between endpoints of elongate portion 91, even in configurations in which elongate portion 91 includes curved portions.

Alternatively or additionally, for some applications, a portion of elongate portion 91 between (a) circuitry portion 100 of PCB 92 and (b) a closest of the one or more electrodes 34 coupled by electrical lead 90 to circuitry 40 has one or more of the following lengths:
at least 50%, no more than 100%, and/or 50%-100% of a length of frame 30, measured parallel to central longitudinal axis 60 of frame 30 (labeled in FIGS. 1A-B),
at least 150%, no more than 1000%, and/or 150%-1000% of a greatest dimension of circuitry portion 100 of PCB 92 (in configurations in which circuitry portion 100 is elongate, the greatest dimension may equal the length long lateral side 105 of circuitry portion 100, labeled in FIG. 3C), and/or
at least 0.5 cm, no more than 6 cm, and/or 0.5-6 cm, such as at least 0.5 cm, no more than 4 cm, and/or 0.5-4 cm (e.g., for configurations in which the closest electrode 34 is disposed proximal (e.g., downstream) of prosthetic leaflets 32 and/or at a proximal (e.g., downstream) half of frame 30), or at least 1.5 cm, no more than 6 cm, and/or 1.5-6 cm (e.g., for configurations in which the closest electrode 34 is disposed distal (e.g., upstream) of the prosthetic leaflets 32).
   All of the above-mentioned lengths of elongate portion 91 are measured in a straight line between endpoints of elongate portion 91, even in configurations in which elongate portion 91 includes curved portions.

For some applications, elongate portion 91 of PCB 92 has one or more of the following widths (perpendicular to a thickness of PCB 92):
at least 0.4, no more than 1.5, and/or 0.4-1.5 mm, and/or
at least 20%, no more than 120%, and/or 20%-120% of a shortest dimension of circuitry portion 100 of PCB 92 perpendicular to a thickness of circuitry portion 100 (in configurations in which circuitry portion 100 is elongate, the shortest dimension may be measured perpendicular to long lateral side 105 of circuitry portion 100, labeled in FIG. 3C).

As mentioned above, electrical lead 90 is coupled to electrode 34. For some applications, electrical lead 90 is coupled to cathode 54, while for other applications, electrical lead 90 is coupled to anode 56. Optionally, more than one electrical lead 90 is integral with elongate portion 91 of PCB 92, in which case a first one of electrical leads 90 may be coupled to cathode 54 and a second one of electrical leads 90 may be coupled to anode 56.

Optionally, a plurality of electrical leads 90 are integral with a corresponding plurality of elongate portions of PCB 92, such as described hereinbelow with reference to FIGS. 3E and 3F.

Optionally, one or more electrodes 34, e.g., one or more cathodes 54 and/or one or more anodes 56, are formed integrally with PCB 92.

Typically, both stent struts 190 and elongate portion 91 of PCB 92 are rectangular in cross section taken perpendicular to respective longitudinal axes of the stent struts and the elongate portion. Typically, electrical lead 90 is also rectangular in cross section, or trapezoidal in cross section. These rectangular cross sections enable flush coupling and/or good crimping of elongate portion 91 to stent struts 190.

For some applications:
stent struts 190 have a thickness of at least 150 microns, such as at least 300 microns; no more than 500 microns; and/or 150-500 microns, such as 300-500 microns,
stent struts 190 have a width of 200-700 microns,
a ratio of the width to the thickness of stent struts 190 is 0.5-2,
electrical lead 90 has a thickness of 5-80 microns, e.g., 50 microns,
electrical lead 90 has a width of 50-300 microns,
a ratio of the width to the thickness of electrical lead 90 is 5-50,
elongate portion 91 of PCB 92 has a thickness of at least 50 microns, no more than 150 microns, and/or 50-150 microns, and/or
elongate portion 91 of PCB 92 has a width of 300-1500 microns, and/or
a ratio of the width to the thickness of elongate portion 91 of PCB 92 is 3-20.

Alternatively or Additionally, for Some Applications:
a ratio of a thickness of stent struts 190 to a thickness of electrical lead 90 is at least 5, no more than 15, and/or 5-15, and/or
a ratio of a thickness of stent struts 190 to a thickness of elongate portion 91 of PCB 92 is at least 2, no more than 5, and/or 2-5.

Elongate portion 91A and/or bifurcation elongate portions 91B of PCB 92, described hereinbelow with reference to FIGS. 3E and/or 3F, may also have the dimensions provided immediately above for elongate portion 91 of PCB 92. Similarly, main portion 90A of electrical lead 90, bifurcation portions 90B of electrical lead 90, electrical lead 90C, electrical lead 90D, and/or electrical lead 90E, described hereinbelow with reference to FIGS. 3E and/or 3F, may also have the dimensions provided immediately above for electrical lead 90.

For some applications, as shown highly schematically in FIG. 3E, PCB 92 comprises a circuitry portion 100, such as an end portion 102 of PCB 92, distinct from elongate portion 91 of PCB 92, and circuitry 40 is coupled to circuitry portion 100 of PCB 92. For some applications, circuitry 40 further comprises (a) tracks 104 (also known as conductive traces) of PCB 92, (b) conductive pads of PCB 92, and (c) electronic components 106 coupled to PCB 92. Elongate portion 91 extends directly from circuitry portion 100 (e.g., end portion 102), and is typically integral with circuitry portion 100 (e.g., end portion 102). (In configurations in which circuitry portion 100 is a mid-portion of PCB 92, rather than end portion 102, PCB 92 extends beyond circuitry portion 100, such as to provide electrical connection to additional elements, e.g., one or more electrodes and/or additional circuitry.) Electrical lead 90 is typically integrally fabricated as a track of elongate portion 91 of PCB 92 in connection with one or more of tracks 104 of PCB 92 that are part of circuitry 40, which obviates the need for a separate connection point between electrical lead 90 and circuitry 40.

For some of these applications, antenna 28 is coupled to circuitry 40 by being coupled to one side of circuitry portion 100 of PCB 92, such as shown in FIGS. 1A-B.

Optionally, elongate portion 91 of PCB 92 is shaped so as to define a plurality of protrusions 98 along elongate portion 91, which inhibit sutures 96 from sliding along elongate portion 91, such that the sutures 96 fix elongate portion 91 of PCB 92 securely to stent struts 190. Typically, protrusions 98 protrude laterally from elongate portion 91 of PCB 92 in a plane defined by PCB 92, either bidirectionally or in a single direction; optionally, some of protrusions 98 protrude bidirectionally and others of protrusions 98 protrude in a single direction, such as shown in the figures. Optionally, as labeled in the enlargement of FIG. 3A, an average distance D of lateral protrusion of protrusions 98 beyond non-protruding portions of elongate portion 91, in a single direction, equals 20%-100% of widths W of elongate portion 91 of PCB 92 at respective locations of the protrusions 98 along elongate portion 91, the average distance D and the widths W measured in the plane defined by PCB 92.

Reference is now made to FIGS. 3E and 3F. In these configurations, elongate portion 91 of PCB 92 is bifurcated, so as to define a main elongate portion 91A and two or more bifurcation elongate portions 91B. By way of example, exactly two bifurcation elongate portions 91B are shown in FIGS. 3E and 3F; in practice, elongate portion 91 may define more than two bifurcation elongate portions.

In some applications, respective electrodes 34, e.g., respective cathodes 54, are coupled to respective bifurcation elongate portions 91B at a respective plurality of angular locations around frame 30.

In some applications, such as shown in FIG. 3E, an electrical lead 90 integral with elongate portion 91 of PCB 92 is bifurcated, so as to define a main portion 90A and two or more bifurcation portions 90B integral with respective bifurcation elongate portions 91B of elongate portion 91 of PCB 92. For example, each of the bifurcation portions 90B of electrical lead 90 may be electrically coupled to a respective electrode 34, e.g., a respective cathode 54, in which case these electrodes are in electrical communication with each other. A separate electrical lead 90C may be provided integral with main elongate portion 91A of elongate portion 91 of PCB 92, in electrical connection with another electrode 34, e.g., an anode 56.

In other applications, such as shown in FIG. 3F, at least two electrical leads 90D and 90E integral with elongate portion 91 of PCB 92. Electrical leads 90D and 90E are partially integral with main elongate portion 91A of elongate portion 91 of PCB 92, and partially integral with respective bifurcation elongate portions 91B of elongate portion 91 of PCB 92. For example, each of electrical leads 90 may be electrically coupled to a respective electrode 34, e.g., a respective cathode 54, in which case these electrodes (e.g., cathodes) are in electrically isolated from each other, and separately electrically connected to circuitry 40. A separate electrical lead 90C may be provided integral with main elongate portion 91A of elongate portion 91 of PCB 92, in electrical connection with another electrode 34, e.g., an anode 56.

For some applications, such as in the configurations described with reference to FIGS. 3E and 3F, circuitry 40 is configured to apply a pacing signal using all of electrodes 34, e.g., all of cathodes 54. For other applications, such as in the configuration described with reference to FIG. 3F, circuitry 40 is configured to apply the pacing signal using fewer than all of electrodes 34, e.g., (a) fewer than all of cathodes 54, for example, using just a single one of cathodes 54, or two or more cathodes 54 of three or more provided cathodes 54, and/or fewer than all of anodes 56, for example, using just a single one of anodes 56, or two or more anodes 56 of three or more provided anodes 56.

Optionally, in configurations in which prosthetic aortic valve 20 comprises a plurality of distal (e.g., upstream) electrodes 34, one or more of the distal (e.g., upstream) electrodes 34 are activated as one or more anodes 56, and one or more other distal (e.g., upstream) electrodes 34 are activated as one or more cathodes 54; in other words, any given distal (e.g., upstream) electrode 34 can be activated as either an anode 56 or a cathode 54.

Alternatively or additionally, optionally, in configurations in prosthetic aortic valve 20 comprises a plurality of proximal (e.g., downstream) electrodes 34, one or more of the proximal (e.g., downstream) electrodes 34 are activated as one or more anodes 56, and one or more other proximal (e.g., downstream) electrodes 34 are activated as one or more cathodes 54; in other words, any given proximal (e.g., downstream) electrode 34 can be activated as either an anode 56 or a cathode 54.

In general, any of electrodes 34 (regardless of their location on frame 30) can be configured as an anode 56 or a cathode 54.

For some applications, circuitry 40 separately activates each of electrodes 34, e.g., cathodes 54 and/or anodes 56, at different times in different combinations, and, based on a determination of which of the electrodes 34 (e.g., cathodes 54, and/or anodes 56 in configurations in which a plurality of anodes 56 are provided) provides the most effective pacing, i.e., the pacing that is successfully obtained using the smallest stimulation voltage. Circuitry 40 uses this most effective combination of electrodes 34, e.g., cathode(s) 54 or anode(s) 56, for future pacing.

For some applications, the determination regarding the most effective pacing is made based on the sensed ECG, as described hereinabove with reference to FIGS. 1A-B and 2, e.g., based on the combination of electrodes that results in the lowest ECG sensing threshold. Alternatively or additionally, for some applications, the determination regarding the most effective pacing is made by selecting the combination of electrodes that yields the lowest power, voltage, or current threshold sufficient for pacing, i.e., successful generation of a cardiac action potential.

In general, circuitry 40 is configured to apply the weakest pacing signal that yields an action potential in the heart. Circuitry 40 may be configured to induce pacing at a set voltage level or alternatively may be set to automatically determine the minimal voltage level of stimulation for a sufficient pacing.

For example, this determination regarding the most effective pacing may be made by circuitry 40 and/or by circuitry of an external control unit, such as external control unit 400, described hereinbelow with reference to FIG. 11. For some applications, this determination is performed (a) only once at the setup of the device immediately after implantation, (b) periodically, e.g., approximately once per day or once per week, and/or (c) before each pacing pulse is applied. An operator may or may not be involved in making the determination.

In some applications, this determination regarding the most effective pacing may be made by activating one or more of the distal (e.g., upstream) electrodes 34 as one or more anodes 56 (rather than as cathodes 54 as labeled in the drawings), and/or activating the distal (e.g., upstream) electrode 34 (or one or more of the distal (e.g., upstream) electrodes if a plurality are provided) as one or more cathodes 54 (rather than as one or more anodes 56 as labeled in the drawings).

Reference is still made to FIGS. 3E-F. It is noted that for clarity of illustration, electrical lead 90 (including main portion 90A and bifurcation portions 90B), electrical lead 90C, electrical lead 90D, and/or electrical lead 90E are shown highly schematically in FIGS. 3E-F. In practice, these electrical leads are typically rectangular in cross section, e.g., having the exemplary dimensions provided hereinabove with reference to FIGS. 1A-B and 3A-D. In addition, these electrical leads may be disposed running alongside one another, such as shown in FIGS. 3E-F, and/or in layers with PCB 92 (configuration not shown), as is known in the PCB art.

Reference is made to FIGS. 3C and 3G. In the configurations shown in these figures, elongate portion 91 extends directly from circuitry portion 100 (e.g., end portion 102), and is typically integral with circuitry portion 100 (e.g., end portion 102). An end portion 103 of elongate portion 91 is bent in a curve over at least a portion of circuitry portion 100, so as to sandwich one or more stent struts 190 between circuitry portion 100 and elongate portion 91, such as shown in FIG. 3G (although not shown in FIG. 3C for the sake of clarity, stent struts 190 are in fact present in prosthetic aortic valve 20). The configurations shown in FIGS. 3C and 3G may optionally be implemented in combination with the other configurations shown herein. (In practice, the one or more stent struts 190 are typically sandwiched more snugly between circuitry portion 100 and elongate portion 91 than shown in FIG. 3G.)

Typically, circuitry portion 100 is disposed radially inward from stent struts 190, end portion 103 of elongate portion 91 is bent in a curve over at least a portion of circuitry portion 100, and the non-curved portion of elongate portion 91 that extends distal (e.g., upstream) from end portion 103 is disposed radially outward from stent struts 190.

For some applications, circuitry portion 100 is elongate, and end portion 103 of elongate portion 91 extends from a long lateral side 105 of circuitry portion 100, such as shown in FIG. 3C, or from a proximal (e.g., downstream) end 107 of circuitry portion 100, such as shown in FIG. 3G. By contrast, if end portion 103 of elongate portion 91 were to instead extend from a distal (e.g., upstream) end of circuitry portion 100, elongate portion 91 might be more likely to be cut during crimping of frame 30. The circumferential width of stent cells 192 diminishes during crimping, while the height of stent cells 192 (in the axial direction) extends during crimping. If the rectangularly cross-sectioned elongate portion 91 were to cross the frame wall when extending from the distal (e.g., upstream) end of circuitry portion 100, elongate portion 91 might be squeezed between two struts during crimping, because the width of elongate portion 91 might be greater than the minimal distance between adjacent nodes or struts during crimping.

Reference is made to FIG. 3H. In this configuration, PCB 92 is shaped so as to define two or more circuitry portions 100 including a first circuitry portion 100A and a second circuitry portion 100B, for example, exactly two circuitry portions 100 (as shown) or three or more circuitry portions 100 (configuration not shown). One or more elongate circuitry-connecting portions 110 of PCB 92 connect the two or more circuitry portions 100. Typically, each of the one or more elongate circuitry-connecting portions 110 comprises one or more electrical leads that are integral with the respective elongate circuitry-connecting portion 110. For some applications, the one or more elongate circuitry-connecting portions 110 extend circumferentially around at least a portion of frame 30.

For some applications, the one or more elongate circuitry-connecting portions 110 are mechanically coupled to some of interconnected stent struts 190 of frame 30, and typically generally run along these stent struts (such that the one or more elongate circuitry-connecting portions 110 may have a zig-zag shape, for example).

Optionally, one of the two or more circuitry portions 100 (e.g., second circuitry portion 100B, as shown) is end portion 102 of PCB 92.

For some applications, circuitry 40 is distributed among the two or more circuitry portions 100, i.e., the two or more circuitry portions 100 comprises respective portions of electronic components of circuitry 40. This may allow the accommodation of circuitry 40 is case a single circuitry portion 100 does not have a sufficient surface area. For some applications, prosthetic aortic valve 20 comprises an energy storage module, e.g., comprising a battery, which is coupled to one of circuitry portions 100.

As used in the present application, including in the claims and Inventive Concepts, “circuitry” means a combination of (a) one or more electronic components 106 and (b) one or more tracks 104 (also known as conductive traces) of a PCB electrically coupled to the one or more electrically components, typically by conductive pads of the PCB. The circuitry may or may not comprise a source of power. The one or more electronic components can be active components (e.g., semiconductor devices, such as integrated circuits, transistors, and/or active diodes); passive components (e.g., electrodes, capacitors, and/or passive diodes); and/or energy storage modules (e.g., comprising a battery). As used in the present application, including in the claims and Inventive Concepts, tracks (also known as traces), electrical leads, wires, and cables are not considered to be electronic components.

Reference is again made to FIGS. 1A-B. The following configuration may be implemented alone or in combination with any of the other configurations described herein, including hereinabove with reference to FIGS. 3A-F and 3H. In this configuration, interconnected stent cells 192 of interconnected stent struts 190 of frame include a first stent cell 170 shaped so as to define:
two peaks 172, consisting of a distal (e.g., upstream) peak 172A and a proximal (e.g., downstream) peak 172B,
two lateral nodes 186, consisting of a left lateral node 186A and a right lateral node 186B,
two left stent struts 176, consisting of (a) a distal (e.g., upstream) left stent strut 176A joined with distal (e.g., upstream) peak 172A and left lateral node 186A, and (b) a proximal (e.g., downstream) left stent strut 176B joined with proximal (e.g., downstream) peak 172B and left lateral node 186A, and
two right stent struts 178, consisting of (a) a distal (e.g., upstream) right stent strut 178A joined with distal (e.g., upstream) peak 172A and right lateral node 186B, and (b) a proximal (e.g., downstream) right stent strut 178B joined with proximal (e.g., downstream) peak 172B and right lateral node 186B.

For example, first stent cell 170 may be located in a proximal (e.g., downstream) half of frame 30, such as shown, e.g., first stent cell 170 may be a proximal (e.g., downstream)-most stent cell (configuration not shown). Alternatively, first stent cell may be located in a distal (e.g., upstream) half of frame 30 (configuration not shown in FIGS. 1A-B), e.g., first stent cell 170 may be a distal (e.g., upstream)-most stent cell (configuration not shown in FIGS. 1A-B).

In this configuration, prosthetic aortic valve 20 comprises an electronic component 150, which is disposed at or near one of peaks 172. For example, electronic component 150 may be part of circuitry 40 (such as shown), may comprise antenna 28 (also such as shown), may comprise an energy storage module, e.g., comprising a battery, or may comprise an electrode 34.

In this configuration, prosthetic aortic valve 20 further comprises coupling material 180, which is shaped so as to define:
a first strip 182A that is mechanically coupled to at least one of left stent struts 176,
a second strip 182B that is mechanically coupled to at least one of right stent struts 178, and
a junction 184, which couples together the first and the second strips 182A and 182B,
   such that first and second strips 182A and 182B together couple electronic component 150 to frame 30 at or near (e.g., within 15 mm of) the one of peaks 172. Using first and second strips 182A and 182B in this arrangement to couple electronic component 150 to frame 30 typically helps stabilize electronic component 150 with respect to frame 30, both during expansion of frame 30 from its compressed elongated state, and during many cardiac cycles after implantation of frame 30.

By way of example and not limitation, in FIGS. 1A-B, first strip 182A is shown mechanically coupled to proximal (e.g., downstream) left stent strut 176B, and second strip 182B is shown mechanically coupled to proximal (e.g., downstream) right stent strut 178B, such that first and second strips 182A and 182B together couple electronic component 150 to frame 30 at or near proximal (e.g., downstream) peak 172B. Alternatively, first strip 182A may be mechanically coupled to distal (e.g., upstream) left stent strut 176A, and second strip 182B may be mechanically coupled to distal (e.g., upstream) right stent strut 178A, such that first and second strips 182A and 182B together couple electronic component 150 to frame 30 at or near distal (e.g., upstream) peak 172A (configuration not shown).

Optionally, first and second strips 182A and 182B are integrally joined at junction 184, e.g., integrally formed from a single piece of material (such as shown); alternatively, first and second strips 182A and 182B comprise discrete pieces of material coupled together at junction 184 (configuration not shown). First strip 182A may be mechanically coupled to either surface of the at least one of left stent struts 176, and second strip 182B may be mechanically coupled to either surface of the at least one of right stent struts 178.

For some applications, first and second strips 182A and 182B together couple electronic component 150 to frame 30 at least partially outside the first stent cell at or near the one of peaks 172.

For some applications, first and second strips 182A and 182B are mechanically coupled to the at least one of left stent struts 176 and the at least one of right stent struts 178, respectively, by stitching.

For some applications, junction 184 of coupling material 180 is mechanically coupled to frame 30 at or near the one of peaks 172, such as by stitching.

For some applications, first strip 182A has length equal to at least 50% of a length of the at least one of left stent struts 176; for example, the length of first strip 182A may be greater than the length of the at least one of left stent struts 176. Alternatively or additionally, for some applications, second strip 182B has length equal to at least 50% of a length of the at least one of right stent struts 178; for example, the length of second strip 182B may be greater than the length of the at least one of right stent struts 178.

For some applications, first strip 182A is mechanically coupled to left lateral node 186A, such as by stitching. Alternatively or additionally, for some applications, second strip 182B is mechanically coupled to right lateral node 186B, such as by stitching.

For some applications, prosthetic aortic valve 20 further comprises an electrical lead, such as electrical lead 90, which is electrically coupled to electronic component 150, and first strip 182A is mechanically coupled to at least a portion of the electrical lead. For some of these applications, first strip 182A comprises electrical insulation, and first strip 182A electrically insulates the at least a portion of the electrical lead. For some applications, first strip 182A comprises an elongate portion of a PCB with which the electrical lead is integral, such as elongate portion 91 of PCB 92.

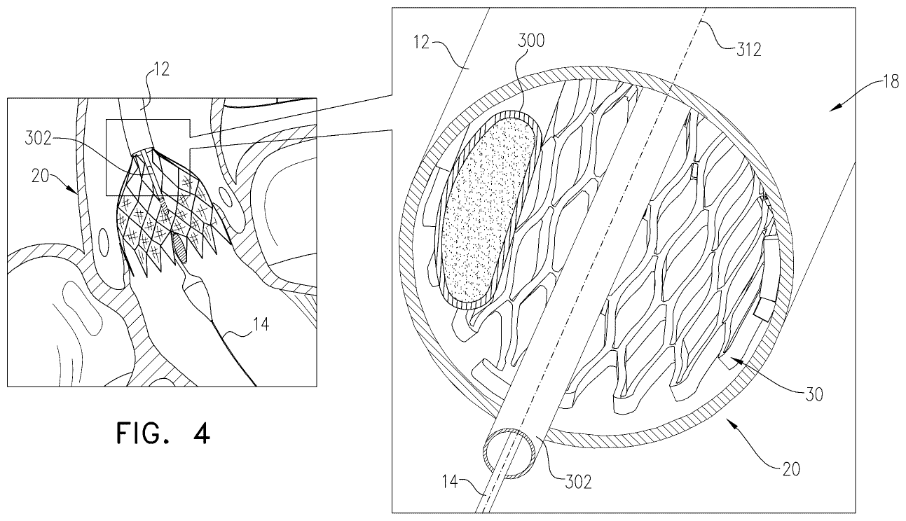

Reference is now made to FIG. 4, which is a schematic illustration of a portion of prosthetic aortic valve 20 in a constrained delivery configuration within delivery sheath 12, in accordance with an application of the present invention.

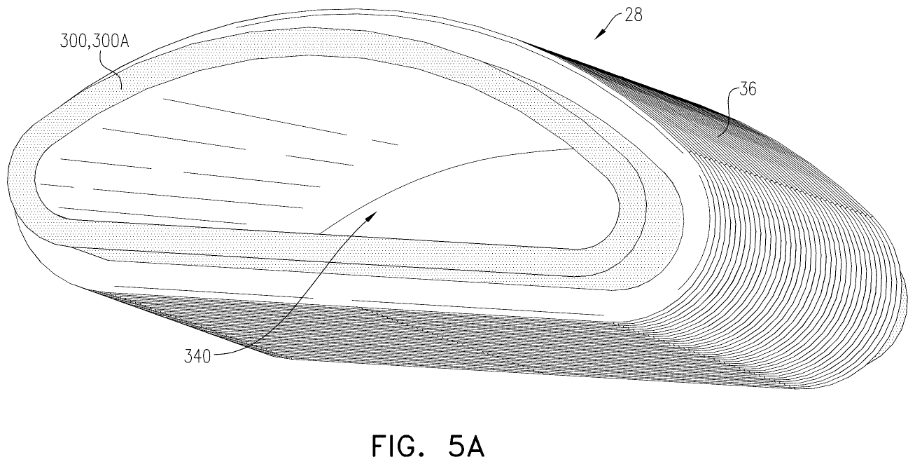

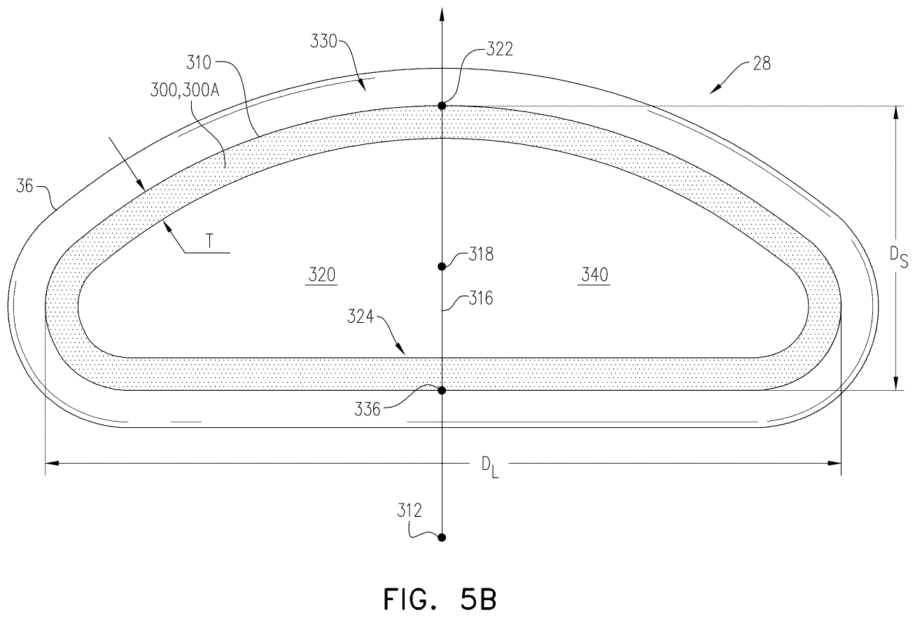

Reference is also made to FIGS. 5A-B, which are schematic illustrations of a magnetic core 300 of antenna 28, in accordance with an application of the present invention. Magnetic core 300 typically comprises a ferrite material, which may or may not be permanently magnetized.

Reference is further made to FIGS. 6A-B, which are schematic illustrations of another configuration of magnetic core 300 of antenna 28, in accordance with an application of the present invention.

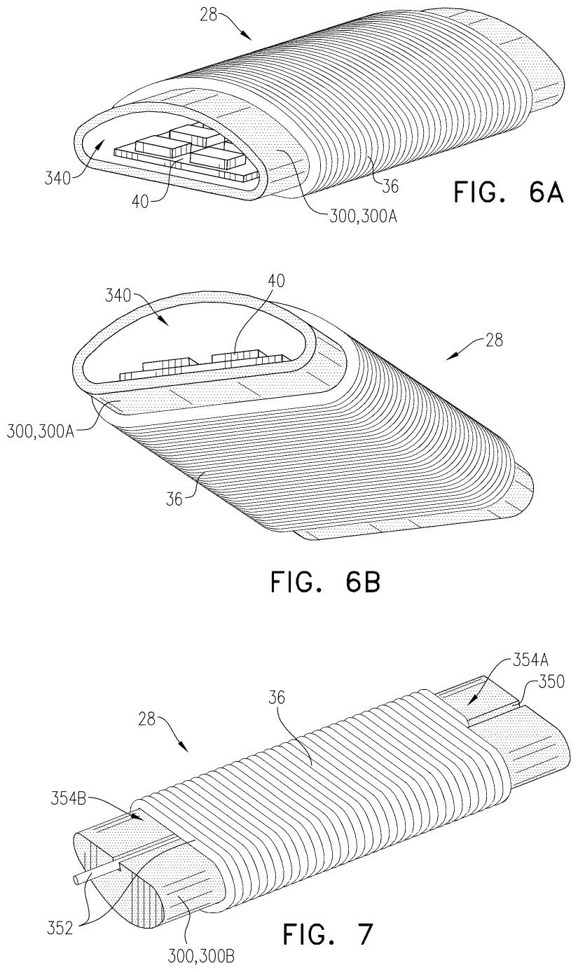

Reference is still further made to FIG. 7, which is a schematic illustration of yet another configuration of magnetic core 300 of antenna 28, in accordance with an application of the present invention.

In these configurations, the one or more prosthetic-valve coils 36 of antenna 28 are wound about magnetic core 300. Magnetic core 300 has a somewhat flattened, non-circular cross section, in order to provide good utilization of the space available on one side of frame 30 between frame 30 and an inner shaft 302 of delivery system 18 when prosthetic aortic valve 20 is in the constrained delivery configuration within delivery sheath 12, such as shown in FIG. 4.

Typically, antenna 28 is mechanically coupled to frame 30 proximal (e.g., downstream) of prosthetic leaflets 32. As a result, magnetic core 300 is disposed at an axial location along prosthetic aortic valve 20 that is devoid of material of prosthetic leaflets 32, because the available space between frame 30 and inner shaft 302 is greater at this axial location than at other axial locations at which prosthetic leaflets 32 are disposed. (Typically, inner shaft 302 is shaped so as to define an internal guidewire channel, through which guidewire 14 passes, as is known in the catheter art.)

For some applications, as labeled in FIGS. 4 and 5B, at at least one location along a length of magnetic core 300, a planar space 320 bound by an outer perimeter 310 of magnetic core 300, in a plane perpendicular to a central longitudinal axis 312 of frame 30 when prosthetic aortic valve 20 is in the constrained delivery configuration, has:
a shorter dimension D_{S}, which is measured along a ray 316 that (i) radiates radially outward from central longitudinal axis 312 and (ii) intersects a centroid 318 defined by planar space 320 bound by outer perimeter 310, and
a longer dimension D_{L}, which is measured perpendicular to the shorter dimension D_{S} in the plane, and is at least 150% of the shorter dimension D_{S}.

(The location of central longitudinal axis 312 is shown schematically and not necessarily to scale in both FIG. 4 and FIG. 5B.)

For some applications, a radially-outward portion 330 of outer perimeter 310 of magnetic core 300, which includes a point 332 on outer perimeter 310 farthest from central longitudinal axis 312, is concavely curved with respect to central longitudinal axis 312. For some of these applications, radially-outward portion 330 of outer perimeter 310 of magnetic core 300 has:
a greatest radius of curvature of at least 1 mm, no more than 5 mm, and/or 1-5 mm, e.g., at least 1.3 mm, no more than 4.5 mm, and/or 1.3-4.5, such as at least 1.5, no more than 3.5 mm, and/or 1.5-3.5, e.g., 2.2 mm.
a greatest radius of curvature of at least 0.3 times the longer dimension D_{L}, no more than 1.6 times the longer dimension D_{L}, and/or 0.3-1.6 times the longer dimension D_{L}., e.g., 0.75-1 times the longer dimension D_{L}.

Alternatively or additionally, for some applications, a radially-inward portion 334 of outer perimeter 310, which includes a point 336 on outer perimeter 310 closest to central longitudinal axis 312, is flat, such as shown in the figures. Alternatively, for some applications, radially-inward portion 334 of outer perimeter 310, which includes one or more points on outer perimeter 310 closest to central longitudinal axis 312, is concavely curved with respect to central longitudinal axis 312 (configuration not shown); optionally, radially-inward portion 334 of outer perimeter 310 has a greatest radius of curvature that is less than a greatest radius of curvature of radially-outward portion 330 of outer perimeter 310.

For some applications, curved radially-outward portion 330 of outer perimeter 310 includes an arcuate portion of a circle. For example, the arcuate portion may have a measure of 45-180 degrees, e.g., 60-180 degrees, such as 60-120 degrees.

Central longitudinal axis 312 intercepts the plane defined by outer perimeter 310 either:
outside planar space 320, in which case point 336 falls along ray 316 between central longitudinal axis 312 and centroid 322, excluding longitudinal axis 312 and centroid 322 (such as shown), or
within planar space 320 (configuration not shown).

Reference is made to FIGS. 5A-B and 6A-B. For some applications, magnetic core 300 is a magnetic core 300, 300A, which is shaped so as to define a cavity 340. Circuitry 40 of prosthetic aortic valve 20 is disposed at least partially within cavity 340, such as entirely within cavity 340. Circuitry 40 is typically electrically coupled to the one or more prosthetic-valve coils 36. Optionally, circuitry 40 comprises circuitry portion 100 of PCB 92, such as described hereinabove with reference to FIG. 3E, and circuitry portion 100 of PCB 92 is disposed at least partially within cavity 340.

For some applications, magnetic core 300 has an average wall thickness T surrounding cavity 340 of 100-500 microns, and/or equal to at least 0.05 (e.g., at least 0.1) times the shorter dimension D_{S}, no more than 0.4 (e.g., no more than 0.3 or no more than 0.2) times the shorter dimension D_{S}, and/or 0.05-0.4 times the shorter dimension D_{S}.

For some applications, the longer dimension D_{L} is at least 175% of the shorter dimension D_{S}, such as at least 200% of the shorter dimension D_{S}. For some applications, the longer dimension D_{L} is no more than 400% of the shorter dimension D_{S}, such as no more than 350% of the shorter dimension D_{S}, e.g., equal to 300% of the shorter dimension D_{S}.

Reference is made to FIG. 7. For some applications, magnetic core 300 is a magnetic core 300, 300B, which is not shaped so as to define a cavity.

For some applications, an external surface of magnetic core 300 is shaped so as to define an axially-oriented groove 350. At least one of prosthetic-valve coils 36 comprises a wire 352, and a straight portion of wire 352 is disposed at least partially within axially-oriented groove 350, so as to pass from a first axial end 354A to a second axial end 354B of the at least one of prosthetic-valve coils 36. (In this context, “axially-oriented” means parallel to a central longitudinal axis of magnetic core 300, or defining an angle of less than 15 degrees with the central longitudinal axis.)

Groove 350 is illustrated in magnetic core 300, 300B by way of example and not limitation, and may be also be implemented in magnetic core 300, 300A, described with reference to FIGS. 5A-B and 6A-B.

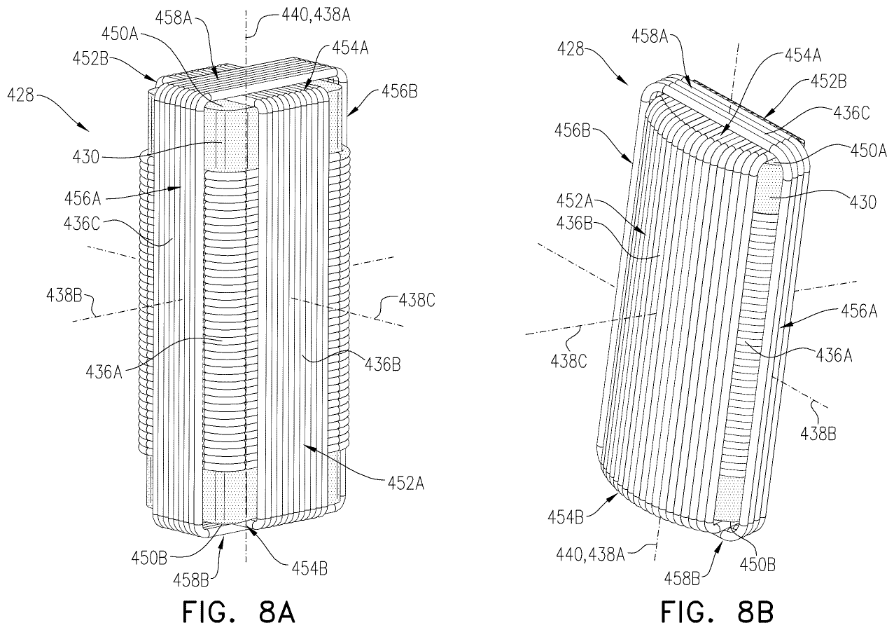

Reference is now made to FIGS. 8A and 8B, which are schematic illustrations of an antenna 428, in accordance with respective applications of the present invention. Antenna 428 may optionally be incorporated into prosthetic aortic valve 20, any of the other prosthetic cardiac valves described herein, or another prosthetic aortic valve, or into another medical device configured to be placed and/or implanted in a body of a subject; alternatively, antenna 428 may implemented independently of any medical device.

Antenna 428 comprises an elongate core 430 and first, second, and third coils 436A, 436B, and 436C, which are wound around elongate core 430 such that:
first coil 436A encircles a first-coil longitudinal axis 438A that coincides with a central longitudinal axis 440 of elongate core 430,
second coil 436B encircles a second-coil longitudinal axis 438B that is perpendicular to first-coil longitudinal axis 438A, and
third coil 436C encircles a third-coil longitudinal axis 438C that is perpendicular to first-coil longitudinal axis 438A and to second-coil longitudinal axis 438B.

For some applications, such as shown in FIG. 8B, antenna 428 implements the techniques of magnetic core 300, described hereinabove with reference to FIGS. 4, 5A-B, and/or 6A-B, while for other applications, antenna 428 does not implement these techniques, such as shown in FIG. 8A. In any event, first, second, and third coils 436A, 436B, and 436C typically are shaped at least in part based on the shape of the external surface of the core around which the coils are wound.

Each of first, second, and third coils 436A, 436B, and 436C comprises an electrically conductive wire coated with electrical insulation.

The longitudinal axes of the coils may or may not be centered within the respective coils.

Typically, first, second, and third coils 436A, 436B, and 436C are electrically isolated from one another and connected to circuitry 40 by separate electrical paths, such that circuitry 40 can separately utilize the coils as appropriate.

Providing three different directions of winding may reduce the dependence on proper orientation of antenna 428 with respect to the antenna of a transmitter/receiver, such as an external transmitter/receiver, e.g., as described hereinbelow with reference to FIG. 2 and/or FIG. 11. Circuitry, such as circuitry 40, may be configured to determine the most effective combination of one or more of the coils for use for transmission of energy and/or data, such as by separately using each of the coils. Alternatively or additionally, circuitry, such as circuitry 40, may be configured to determine an orientation of antenna 428 with respect to the antenna of a transmitter/receiver, such as an external transmitter/receiver, e.g., as described hereinbelow with reference to FIG. 2 and/or FIG. 11, based on the relative strengths of the signals in each of the coils. The circuitry may perform either of the above-mentioned determinations either during a pre-procedural calibration procedure or during use of antenna 428.

For some applications, second and third coils 436B and 436C cross each other at one or both longitudinal ends 450A and 450B of elongate core 430. Although longitudinal ends 450A and 450B are shown as flat in FIGS. 8A and 8B, the ends may alternatively define curved surfaces, such as convex curved surfaces.

Second coil 436B typically has (a) two longer sides 452A and 452B, which may or may not have the same lengths as each other, and (b) two shorter sides 454A and 454B, which may or may not have the same lengths as each other. For some applications, the two longer sides 452A and 452B are parallel to central longitudinal axis 440 of elongate core 430, or define an angle of less than 10 degrees with respect to central longitudinal axis 440 of elongate core 430, such as less than 5 degrees.

For some applications, the two longer sides 452A and 452B cross first coil 436A at a plurality of first locations, and define angles of 75-90 degrees, such as 80-90 degrees, e.g., 85-90 degrees with first coil 436A at each of the plurality of first locations.

Similarly, third coil 436C typically has (a) two longer sides 456A and 456B, which may or may not have the same lengths as each other and/or as longer sides 452A and 452B, and (b) two shorter sides 458A and 458B, which may or may not have the same lengths as each other and/or as shorter sides 454A and 454B. For some applications, the two longer sides 456A and 456B are parallel to central longitudinal axis 440 of elongate core 430, or define an angle of less than 10 degrees with respect to central longitudinal axis 440 of elongate core 430, such as less than 5 degrees.

For some applications, the two longer sides 456A and 456B cross first coil 436A at a plurality of second locations, and define angles of 85-90 degrees with first coil 436A at each of the plurality of second locations.

Optionally, elongate core 430 of antenna 428 is shaped so as to define axially-oriented groove 350, described hereinabove with reference to FIG. 7.

Reference is now made to FIG. 9, which is a schematic illustration of a prosthetic atrioventricular valve 520, in accordance with an application of the present invention. Other than as described hereinbelow, prosthetic atrioventricular valve 520 may be generally similar to prosthetic aortic valve 20, described hereinabove with reference to FIG. 1A-3H, and may implement any of the features thereof, mutatis mutandis. Prosthetic atrioventricular valve 520 may also optionally implement any of the features described hereinabove with reference to FIGS. 4-8B, mutatis mutandis.

Prosthetic atrioventricular valve 520 may be a prosthetic mitral valve or a prosthetic tricuspid valve. Typically, but not necessarily, prosthetic atrioventricular valve 520 has a shorter length than the configurations of prosthetic aortic valve 20 shown in the figures. Optionally, prosthetic atrioventricular valve 520 implements any techniques known in the art for transcatheter prosthetic atrioventricular valves. For example, prosthetic atrioventricular valve 520 may implement techniques described in PCT Publication WO 2022/118316 to Albitov et al., US Patent Application Publication 2015/0328000 to Ratz et al., U.S. Pat. No. 10,299,927 to McLean et al., and/or U.S. Pat. No. 7,510,575 to Spenser, all of which are incorporated herein by reference.

Prosthetic atrioventricular valve 520 comprises a frame 530 and a plurality of prosthetic leaflets coupled to frame 530 so as to allow blood flow in a downstream direction and inhibit blood flow in an upstream direction when prosthetic atrioventricular valve 520 is in an expanded deployment configuration, such as shown in FIG. 9. For clarity of illustration, the prosthetic leaflets are not shown in FIG. 9; in practice they are provided, and may be similar to prosthetic leaflets 32, described hereinabove, mutatis mutandis, and/or may implement any techniques of prosthetic leaflets of prosthetic atrioventricular valves known in art, including, but not limited to, the techniques described in the patent publications incorporated hereinabove.

Prosthetic atrioventricular valve 520 has an upstream end 522 and a downstream end 524. Upstream end 522 may also be a proximal end 26 and downstream end 524 may also be a distal end 27, for example because proximal end 26 may be disposed in distal end portion 31 of delivery sheath 12 more proximally than distal end 27; in other words, proximal end 26 is closer to proximal end portion 29 of delivery sheath 12 than is distal end 27. For some applications, such as shown, proximal end 26 is configured to be coupled to delivery system 18 (e.g., shaped so as to define delivery-tool-coupling tabs 220, which are configured to removably couple frame 30, and thus prosthetic atrioventricular valve 520, to delivery system 18, e.g., to a delivery shaft of delivery system 18, such as described herein). For other applications (configuration not shown), distal end 27 is configured to be coupled to delivery system 18, such as to a capsule of the distal end portion 31 of delivery sheath 12, such as described hereinabove.

In some applications of the present invention, prosthetic atrioventricular valve 520 implements any of the techniques described hereinabove with reference to FIGS. 1A-4 for prosthetic aortic valve 20. In some of these techniques, “proximal” features of prosthetic aortic valve 20 are described as “downstream” features, and “distal” features of prosthetic aortic valve 20 are described as “upstream” features. Typically, in prosthetic atrioventricular valve 520 these directions are the opposite, such that “proximal” features of atrioventricular valve 520 are “upstream” features, and “distal” features of atrioventricular valve 520 are “downstream” features.

Reference is now made to FIG. 10, which is a schematic illustration of a frame 630 of a prosthetic cardiac valve 620 when prosthetic cardiac valve 620 is in an expanded deployment configuration, in accordance with an application of the present invention. Frame 630 may, for example, comprise frame 30 of prosthetic aortic valve 20, or frame 530 of prosthetic atrioventricular valve 520. For clarity of illustration, the portion of frame 630 is shown laid flat; in practice, the frame is curved around central longitudinal axis 60 when prosthetic cardiac valve 620 is in the expanded deployment configuration. In addition, although the description below and FIG. 10 relate to antenna 28, the same techniques are applicable to antenna 428.

In the following description, “downstream” is provided as an example of “proximal,” and “upstream” is provided as an example of “distal”; this example relates to configurations in which frame 630 comprises frame 30 of prosthetic aortic valve 20. In configurations in which frame 630 comprises frame 530 of prosthetic atrioventricular valve 520, all of these exemplary directions would be reversed, i.e., “upstream” would be an example of “proximal,” and “downstream” would be an example of “distal.” Both examples are within the scope of the present invention.

In the following description, and as labeled in FIG. 10, antenna 28 is shown coupled to frame 630 near a proximal end of frame 630. In an alternative application of the present invention, antenna 28 is instead coupled to frame 630 near a distal end of frame 630, in which case all of the mentions of proximal and distal would be reversed in the following description and in the corresponding claims and Inventive Concepts.

For some applications, antenna 28 is approximately aligned with a proximal (e.g., downstream) end of frame 630, between circumferentially adjacent first and second proximal (e.g., downstream)-most stent cells 206A and 206B of interconnected stent cells 192. This location strikes a balance between the benefit of avoiding attenuation by the metal scaffold of frame 630 and the operational constraints of not interfering with the interface between one or more proximally-extending delivery-tool-coupling tabs 220 of frame 630 and delivery system 18. In an experiment conducted by one of the inventors, it was found that the relative attenuation when antenna 28 was disposed as shown in FIG. 10 was less than half of the relative attenuation when antenna 28 was disposed at the same axial location but instead within one of the cells.

As described above with reference to FIGS. 1A-B and 2, frame 630 defines central longitudinal axis 60 when prosthetic cardiac valve 620 is in the expanded deployment configuration, and frame 630 comprises interconnected stent struts 190 arranged so as to define interconnected stent cells 192.

For some applications, first and second proximal (e.g., downstream) peaks 204A and 204B respectively defined by circumferentially adjacent first and second proximal (e.g., downstream)-most stent cells 206A and 206B of interconnected stent cells 192 are located at respective first and second peak angular locations 208A and 208B about central longitudinal axis 60 of frame 630. As used in the present application, including in the claims and Inventive Concepts, an “angular location” is a location on frame 630 at a particular location around central longitudinal axis 60, i.e., at a particular “o'clock” with respect to central longitudinal axis 60.

Antenna 28 is mechanically coupled to frame 630 such that:
a centroid 212 of antenna 28 is at an antenna angular location 214 about central longitudinal axis 60, antenna angular location 214 between first and second peak angular locations 208A and 208B, and
a proximal (e.g., downstream)-most point 216 of antenna 28 is axially disposed between (i) 5 mm proximal (e.g., downstream) of first and second proximal (e.g., downstream) peaks 204A and 204B (schematically indicated by a line 240A) and (ii) 5 mm distal (e.g., upstream) of first and second proximal (e.g., downstream) peaks 204A and 204B (schematically indicated by a line 240B).

Downstream-most point 216 of antenna 28 may be defined by a core of the antenna, such as magnetic core 300, such as shown in FIG. 10, or by one of the coils of the antenna, such as in the configuration of antenna 428, described hereinabove with reference to FIGS. 8A and 8B.

For some applications, antenna 28 is mechanically coupled to frame 630 such that proximal (e.g., downstream)-most point 216 of antenna 28 is axially disposed between (i) 3 mm proximal (e.g., downstream) of first and second proximal (e.g., downstream) peaks 204A and 204B and (ii) 5 mm distal (e.g., upstream) of first and the second proximal (e.g., downstream) peaks 204A and 204B (schematically indicated by a line 240B). For example, antenna 28 may be mechanically coupled to frame 630 such that proximal (e.g., downstream)-most point 216 of antenna 28 is axially disposed between (i) an axial location of first and second proximal (e.g., downstream) peaks 204A and 204B and (ii) 5 mm distal (e.g., upstream) of first and the second proximal (e.g., downstream) peaks 204A and 204B (schematically indicated by a line 240B), e.g., axially disposed at a same axial location as first and second proximal (e.g., downstream) peaks 204A and 204B, such as shown in FIG. 10.

For some applications, antenna 28 is mechanically coupled to frame 630 such that proximal (e.g., downstream)-most point 216 of antenna 28 is axially disposed between (i) 5 mm proximal (e.g., downstream) of first and second proximal (e.g., downstream) peaks 204A and 204B (schematically indicated by a line 240A) and (ii) 3 mm distal (e.g., upstream) of first and the second proximal (e.g., downstream) peaks 204A and 204B. For some of these applications, antenna 28 is mechanically coupled to frame 630 such that proximal (e.g., downstream)-most point 216 of antenna 28 is axially disposed between (i) 3 mm proximal (e.g., downstream) of first and second proximal (e.g., downstream) peaks 204A and 204B and (ii) 3 mm distal (e.g., upstream) of first and the second proximal (e.g., downstream) peaks 204A and 204B.

For some applications, frame 630 further comprises one or more delivery-tool-coupling tabs 220, disposed proximal (e.g., downstream) of stent cells 192, and shaped so as to define respective distal (e.g., upstream)-facing edges 222. The one or more delivery-tool-coupling tabs 220 are configured to removably couple frame 630, and thus prosthetic cardiac valve 620, to delivery system 18, e.g., to a delivery shaft of delivery system 18.

For some of these applications, antenna 28 is mechanically coupled to frame 630 such that:
centroid 212 of antenna 28 is at antenna angular location 214 about central longitudinal axis 60, antenna angular location 214 between first and second peak angular locations 208A and 208B, and
downstream-most point 216 of antenna 28 is axially disposed between (i) an axial position of distal (e.g., upstream)-facing edges 222 of delivery-tool-coupling tabs 220 (schematically indicated by a line 240C) and (ii) 5 mm distal (e.g., upstream) of first and second proximal (e.g., downstream) peaks 204A and 204B (schematically indicated by a line 240B) (it is noted that antenna 28 is typically circumferentially (angularly) offset from delivery-tool-coupling tabs 220, such as shown).

For some applications, antenna 28 is mechanically coupled to frame 630 such that proximal (e.g., downstream)-most point 216 of antenna 28 is axially disposed between (i) 2 mm distal (e.g., upstream) of distal (e.g., upstream)-facing edges 222 of delivery-tool-coupling tabs 220 and (ii) 5 mm distal (e.g., upstream) of first and second proximal (e.g., downstream) peaks 204A and 204B (schematically indicated by a line 240B).

For some applications, antenna 28 is mechanically coupled to frame 630 such that proximal (e.g., downstream)-most point 216 of antenna 28 is axially disposed between (i) 5 mm distal (e.g., upstream) of distal (e.g., upstream)-facing edges 222 of delivery-tool-coupling tabs 220 and (ii) 3 mm distal (e.g., upstream) of first and second proximal (e.g., downstream) peaks 204A and 204B.

The locations of lines 240A, 240B, and 240C are shown in FIG. 10 by way of example and not limitation, and are based on exemplary approximate dimensions and shapes of interconnected stent struts 190 and interconnected stent cells 192.

For some applications, first and second proximal (e.g., downstream)-most stent cells 206A and 206B are joined at cell junction 210, and antenna 28 is mechanically coupled to frame 630 at least in part by being mechanically coupled to cell junction 210. For some of these applications, a distal (e.g., upstream)-most point 270 of antenna 28 coincides with, or is no more than a distance distal (e.g., upstream) of, the cell junction, the distance equal to 30% of a length of antenna 28, such as 20% of the length of antenna 28, the distance and the length measured parallel to central longitudinal axis 60 of frame 630.

First and second peak angular locations 208A and 208B are angularly offset by a peak-to-peak angular offset α (alpha). First peak angular location 208A and antenna angular location 214 are angularly offset by a peak-to-antenna angular offset β (beta). For some applications, peak-to-antenna angular offset α (alpha) equals 25%-75% of peak-to-peak angular offset β (beta), e.g., 50%, as shown in FIG. 10.

For some applications, a width of antenna 28, measured in a peak-to-peak direction, equals 10%-60% of peak-to-peak angular offset α (alpha), e.g., 10%-30%, e.g., 15% of α (alpha).

A peak height H equals a distance between a proximal (e.g., downstream)-most point 272 of first proximal (e.g., downstream) peak 204A and cell junction 210, measured parallel to central longitudinal axis 60 of frame 630. For some applications, a length of antenna 28 equals 30%-150% of peak height H, such as 80-120%, e.g., 100%, of peak height H, the length and the peak height measured parallel to central longitudinal axis 60 of frame 630.

Reference is now made to FIG. 11, which is a schematic illustration of an external control unit 400 of valve prosthesis system 10, in accordance with an application of the present invention. A prosthetic cardiac valve, such as prosthetic aortic valve 20 or prosthetic atrioventricular valve 520, is configured to be delivered to a native cardiac valve of a patient in a constrained delivery configuration within delivery sheath 12 using guidewire 14.

External control unit 400 is configured to be disposed outside a body of the patient, and comprises:
a housing 410, which is shaped so as to define a guidewire-receiving channel 412;
a rapid-pacing user control 414; and
external-unit control circuitry 418.

Reference is again made to FIG. 2. Typically, an external system is provided that is configured to be disposed outside a body of the patient. The external system comprises an external control unit 700, which may, for example, comprise external control unit 400, described hereinabove with reference to FIG. 11.

For some applications, the external system further comprises an external transmitter and/or receiver, which optionally comprises an external coil 420, which is highly schematically illustrated in FIG. 2. For example, external coil 420 may be configured to be placed around the subject's chest, such as schematically shown in FIG. 2, or placed against the chest without surrounding the chest, such as against the sternum (configuration not shown). The external transmitter and/or receiver is configured to wirelessly transfer energy to at least one of the one or more prosthetic-valve coils 36, such as by driving external coil 420 to wirelessly transfer the energy to at least one of the one or more prosthetic-valve coils 36 by inductive coupling. For example, the external transmitter may transmit RF energy at a frequency of 2-300 MHz, e.g., 6.78 MHz.

Reference is again made to FIG. 11. As described hereinabove with reference to FIGS. 1A-B and 2, for some applications, circuitry 40 is configured to apply both regular pacing and rapid pacing. For example, the rapid pacing may be applied during an invasive structural heart procedure, such as an implantation procedure, such as a TAVR-in-TAVR procedure in which the first TAVR comprises prosthetic aortic valve 20, and a portion of the regular pacing may be applied temporarily while the patient is hospitalized after implantation of prosthetic aortic valve 20. External control unit 400 may be provided for controlling both the regular pacing and the rapid pacing. (When the patient is discharged from the hospital, an external control unit is typically provided having fewer or no user controls accessible by the patient.) Because the user or healthcare works may have access to external control unit 400, it is desirable to prevent accidental activation of rapid pacing after completion of the implantation procedure.

For some applications, external-unit control circuitry 418 is configured to:
drive an external transmitter to wirelessly transfer energy to at least one of the one or more prosthetic-valve coils 36, such as for powering regular pacing (for example, by driving an energy-transmission coil of the external transmitter to wirelessly transfer the energy by inductive coupling), and
only upon activation of rapid-pacing user control 414 and when guidewire 14 is disposed within guidewire-receiving channel 412 of housing 410, drive prosthetic aortic valve 20 or prosthetic atrioventricular valve 520 to apply rapid pacing using cathode 54 and anode 56.

To this end, external control unit 400 comprises a sensor, configured to sense whether guidewire 14 is disposed within guidewire-receiving channel 412 of housing 410.

This feature may serve as a safety feature, which restricts application of the rapid pacing to a transcatheter or surgical cardiovascular operation by a certified medical interventionalist.

The techniques described herein for prosthetic aortic valve 20 may be alternatively used, mutatis mutandis, for non-aortic prosthetic valves, such as prosthetic atrioventricular valves (prosthetic mitral valves or prosthetic tricuspid valves), such as described hereinabove with reference to FIG. 9.

In an embodiment, techniques and apparatus described in one or more of the following patents and/or applications, which are assigned to the assignee of the present application and are incorporated herein by reference, are combined with techniques and apparatus described herein:
U.S. Pat. No. 10,543,083 to Gross
European Patent Application Publication EP 3508113 A1 to Gross
U.S. Pat. No. 10,835,750 to Gross
U.S. Pat. No. 11,013,597 to Gross
PCT Publication WO 2021/140507 to Gross
PCT Publication WO 2021/224904 to Gross
U.S. Pat. No. 11,065,451 to Gross
U.S. Pat. No. 11,291,844 to Gross
PCT Publication WO 2022/149130 to Gross
U.S. patent application Ser. No. 18/452,216, filed Aug. 18, 2023, which issued as U.S. Pat. No. 11,975,203 to Gross et al.
U.S. patent application Ser. No. 18/607,638, filed Mar. 18, 2024

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction; and
an antenna, which is mechanically coupled to the frame proximal of the prosthetic leaflets, and which comprises one or more prosthetic-valve coils,
wherein first and second proximal peaks respectively defined by circumferentially adjacent first and second proximal-most stent cells of the interconnected stent cells are located at respective first and second peak angular locations about the central longitudinal axis of the frame, and
wherein the antenna is mechanically coupled to the frame such that (a) a centroid of the antenna is at an antenna angular location about the central longitudinal axis, the antenna angular location between the first and the second peak angular locations, and (b) a proximal-most point of the antenna is axially disposed between (i) 5 mm proximal of the first and the second proximal peaks and (ii) 5 mm distal of the first and the second proximal peaks.

2. The prosthetic cardiac valve according to claim 1, wherein the antenna is mechanically coupled to the frame such that the proximal-most point of the antenna is axially disposed between (i) 3 mm proximal of the first and the second proximal peaks and (ii) 5 mm distal of the first and the second proximal peaks.

3. The prosthetic cardiac valve according to any one of claims 1-2,
wherein the prosthetic cardiac valve is a prosthetic aortic valve,
wherein the antenna is mechanically coupled to the frame downstream of the prosthetic leaflets,
wherein the circumferentially adjacent first and second proximal-most stent cells are circumferentially adjacent first and second downstream-most stent cells, respectively,
wherein the first and the second proximal peaks are first and second downstream peaks respectively defined by the circumferentially adjacent first and second downstream-most stent cells, and
wherein the proximal-most point of the antenna is a downstream-most point of the antenna that is axially disposed between (i) 5 mm downstream of the first and the second downstream peaks and (ii) 5 mm upstream of the first and the second downstream peaks.

4. The prosthetic cardiac valve according to any one of claims 1-2,
wherein the prosthetic cardiac valve is a prosthetic atrioventricular valve,
wherein the antenna is mechanically coupled to the frame upstream of the prosthetic leaflets,
wherein the circumferentially adjacent first and second proximal-most stent cells are circumferentially adjacent first and second upstream-most stent cells, respectively,
wherein the first and the second proximal peaks are first and second upstream peaks respectively defined by the circumferentially adjacent first and second upstream-most stent cells, and
wherein the proximal-most point of the antenna is an upstream-most point of the antenna that is axially disposed between (i) 5 mm upstream of the first and the second upstream peaks and (ii) 5 mm downstream of the first and the second upstream peaks.

5. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises:
interconnected stent struts arranged so as to define interconnected stent cells; and
one or more delivery-tool-coupling tabs, disposed proximal of the stent cells, and shaped so as to define respective distal-facing edges;
a plurality of prosthetic leaflets coupled to the frame so as to allow blood flow in a downstream direction and inhibit blood flow in an upstream direction; and
an antenna, which is mechanically coupled to the frame proximal of the prosthetic leaflets, and which comprises one or more prosthetic-valve coils,
wherein first and second proximal peaks respectively defined by circumferentially adjacent first and second proximal-most stent cells of the interconnected stent cells are located at respective first and second peak angular locations about the central longitudinal axis of the frame, and
wherein the antenna is mechanically coupled to the frame such that (a) a centroid of the antenna is at an antenna angular location about the central longitudinal axis, the antenna angular location between the first and the second peak angular locations, and (b) a proximal-most point of the antenna is axially disposed between (i) an axial position of the distal-facing edges of the delivery-tool-coupling tabs and (ii) 5 mm distal of the first and the second proximal peaks.

6. A valve prosthesis system comprising the prosthetic cardiac valve according to claim 5, the valve prosthesis system further comprising a delivery system, which comprises a delivery shaft that is removably couplable to the one or more delivery-tool-coupling tabs.

7. The prosthetic cardiac valve according to claim 5,
wherein the prosthetic cardiac valve is a prosthetic aortic valve,
wherein the antenna is mechanically coupled to the frame downstream of the prosthetic leaflets,
wherein the one or more delivery-tool-coupling tabs are disposed downstream of the stent cells, and shaped so as to define respective upstream-facing edges,
wherein the circumferentially adjacent first and second proximal-most stent cells are circumferentially adjacent first and second downstream-most stent cells, respectively,
wherein the first and the second proximal peaks are first and second downstream peaks respectively defined by the circumferentially adjacent first and second downstream-most stent cells, and
wherein the proximal-most point of the antenna is a downstream-most point of the antenna that is axially disposed between (i) an axial position of the upstream-facing edges of the delivery-tool-coupling tabs and (ii) 5 mm upstream of the first and the second downstream peaks.

8. The prosthetic cardiac valve according to claim 5,
wherein the prosthetic cardiac valve is a prosthetic atrioventricular valve,
wherein the antenna is mechanically coupled to the frame upstream of the prosthetic leaflets,
wherein the one or more delivery-tool-coupling tabs are disposed upstream of the stent cells, and shaped so as to define respective downstream-facing edges,
wherein the circumferentially adjacent first and second proximal-most stent cells are circumferentially adjacent first and second upstream-most stent cells, respectively,
wherein the first and the second proximal peaks are first and second upstream peaks respectively defined by the circumferentially adjacent first and second upstream-most stent cells, and
wherein the proximal-most point of the antenna is an upstream-most point of the antenna that is axially disposed between (i) an axial position of the downstream-facing edges of the delivery-tool-coupling tabs and (ii) 5 mm downstream of the first and the second upstream peaks.

9. The prosthetic cardiac valve according to any one of claims 1 and 5, wherein the antenna comprises a magnetic core around which are wound the one or more prosthetic-valve coils.

10. The prosthetic cardiac valve according to any one of claims 1 and 5,
wherein the first and the second proximal-most stent cells are joined at a cell junction,
wherein the first proximal-most stent cell comprises a right proximal strut of the interconnected stent struts, the right proximal strut extending between the cell junction and a first proximal peak defined by the first proximal-most stent cell,
wherein the second proximal-most stent cell comprises a left proximal strut of the interconnected stent struts, the left proximal strut extending between the cell junction and a second proximal peak defined by the second proximal-most stent cell,
wherein a flexible sheet is mechanically coupled to the right and the left proximal struts, and
wherein the antenna is mechanically coupled to the frame at least in part by being mechanically coupled to the flexible sheet between the right and the left proximal struts.

11. The prosthetic cardiac valve according to any one of claims 1 and 5, wherein the first and the second proximal-most stent cells are joined at a cell junction, and the antenna is mechanically coupled to the frame at least in part by being mechanically coupled to the cell junction.

12. The prosthetic cardiac valve according to claim 11, wherein a distal-most point of the antenna coincides with, or is no more than a distance distal of, the cell junction, the distance equal to 30% of a length of the antenna, the distance and the length measured parallel to the central longitudinal axis of the frame.

13. The prosthetic cardiac valve according to any one of claims 1 and 5,
wherein the first and the second peak angular locations are angularly offset by a peak-to-peak angular offset,
wherein the first peak angular location and the antenna angular location are angularly offset by a peak-to-antenna angular offset, and
wherein the peak-to-antenna angular offset equals 25%-75% of the peak-to-peak angular offset.

14. The prosthetic cardiac valve according to any one of claims 1 and 5, wherein the proximal-most point of the antenna is axially disposed between 5 mm proximal of the first and the second proximal peaks and 5 mm distal of the first and the second proximal peaks.

15. The prosthetic cardiac valve according to any one of claims 1 and 5,
wherein the circumferentially adjacent first and second proximal-most stent cells are joined at a cell junction,
wherein a peak height equals a distance between a proximal-most point of the first proximal peak and the cell junction, measured parallel to the central longitudinal axis of the frame, and
wherein a length of the antenna equals 30%-150% of the peak height, the length and the peak height measured parallel to the central longitudinal axis of the frame.

16. The prosthetic cardiac valve according to any one of claims 1 and 5,
wherein the first and the second peak angular locations are angularly offset by a peak-to-peak angular offset, and
wherein a width of the antenna, measured in a peak-to-peak direction, equals 10%-60% of the peak-to-peak angular offset.

17. The prosthetic cardiac valve according to any one of claims 1 and 5, further comprising:
a cathode and an anode, which are mechanically coupled to the frame; and
circuitry, which is electrically coupled to the cathode, the anode, and the one or more prosthetic-valve coils.

18. A valve prosthesis system comprising the prosthetic cardiac valve according to any one of claims 1 and 5, the valve prosthesis system further comprising an external unit, wherein the external unit is configured to be disposed outside a body of the patient, and comprises:
an energy-transmission coil; and
external-unit control circuitry, which is configured to drive the energy-transmission coil to wirelessly transfer energy to at least one of the one or more prosthetic-valve coils by inductive coupling.

19. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction;
an antenna, which is mechanically coupled to the frame proximal of the prosthetic leaflets, and which comprises one or more prosthetic-valve coils; and
a flexible sheet,
wherein circumferentially adjacent first and second proximal-most stent cells of the interconnected stent cells are joined at a cell junction,
wherein the first proximal-most stent cell comprises a right proximal strut of the interconnected stent struts, the right proximal strut extending between the cell junction and a first proximal peak defined by the first proximal-most stent cell,
wherein the second proximal-most stent cell comprises a left proximal strut of the interconnected stent struts, the left proximal strut extending between the cell junction and a second proximal peak defined by the second proximal-most stent cell,
wherein the flexible sheet is mechanically coupled to the right and the left proximal struts, and
wherein the antenna is mechanically coupled to the frame at least in part by being mechanically coupled to the flexible sheet between the right and the left proximal struts.

20. The prosthetic cardiac valve according to claim 19, wherein the antenna is mechanically coupled to the frame at least in part by being mechanically coupled to the cell junction.

21. The prosthetic cardiac valve according to claim 19, wherein the antenna is mechanically coupled to the flexible sheet by stitching.

22. The prosthetic cardiac valve according to claim 19, wherein the flexible sheet is mechanically coupled to the right and the left proximal struts by stitching.

23. The prosthetic cardiac valve according to claim 19, wherein the antenna comprises a magnetic core around which are wound the one or more coils.

24. The prosthetic cardiac valve according to claim 19, wherein the flexible sheet is coupled only to one or more of the interconnected stent struts of each of the first and the second proximal-most stent cells, and not to any of the interconnected stent struts of other stent cells of the frame.

25. The prosthetic cardiac valve according to any one of claims 19-24, wherein the frame further comprises one or more delivery-tool-coupling tabs, disposed proximal of the stent cells.

26. A valve prosthesis system comprising the prosthetic cardiac valve according to any one of claims 19-25, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

27. The prosthetic cardiac valve according to any one of claims 19-25,
wherein the prosthetic cardiac valve is a prosthetic aortic valve,
wherein the antenna is mechanically coupled to the frame downstream of the prosthetic leaflets,
wherein the circumferentially adjacent first and second proximal-most stent cells are circumferentially adjacent first and second downstream-most stent cells,
wherein the right proximal strut is a right downstream strut of the interconnected stent struts,
wherein the first proximal peak defined by the first proximal-most stent cell is a first downstream peak defined by the first downstream-most stent cell,
wherein the left proximal strut is a left downstream strut of the interconnected stent struts,
wherein the second proximal peak defined by the second proximal-most stent cell is s second downstream peak defined by the second downstream-most stent cell,
wherein the flexible sheet is mechanically coupled to the right and the left downstream struts, and
wherein the antenna is mechanically coupled to the frame at least in part by being mechanically coupled to the flexible sheet between the right and the left downstream struts.

28. The prosthetic cardiac valve according to any one of claims 19-25,
wherein the prosthetic cardiac valve is a prosthetic atrioventricular valve,
wherein the antenna is mechanically coupled to the frame upstream of the prosthetic leaflets,
wherein the circumferentially adjacent first and second proximal-most stent cells are circumferentially adjacent first and second upstream-most stent cells,
wherein the right proximal strut is a right upstream strut of the interconnected stent struts,
wherein the first proximal peak defined by the first proximal-most stent cell is a first upstream peak defined by the first upstream-most stent cell,
wherein the left proximal strut is a left upstream strut of the interconnected stent struts,
wherein the second proximal peak defined by the second proximal-most stent cell is s second upstream peak defined by the second upstream-most stent cell,
wherein the flexible sheet is mechanically coupled to the right and the left upstream struts, and
wherein the antenna is mechanically coupled to the frame at least in part by being mechanically coupled to the flexible sheet between the right and the left upstream struts.

29. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells, wherein distal ones of the stent cells are located in a distal half of the frame and define respective distal peaks;
a plurality of prosthetic leaflets coupled to the frame so as to allow blood flow in a downstream direction and inhibit blood flow in an upstream direction;
an electrode, which is disposed at or near a distal peak of one of the distal stent cells, wherein first and second distal stent struts of the one of the distal stent cells are joined at the distal peak; and
coupling material, which is shaped so as to define:
(a) a first strip that is mechanically coupled to the first distal stent strut,
(b) a second strip that is mechanically coupled to the second distal stent strut, and
(c) a junction, which couples together the first and the second strips,
such that the first and the second strips together couple the electrode to the frame at or near the distal peak.

30. The prosthetic cardiac valve according to claim 29, wherein the distal ones of the stent cells are distal-most ones of the stent cells, and the one of the distal stent cells is one of the distal-most stent cells.

31. The prosthetic cardiac valve according to claim 29, wherein the first and the second strips are mechanically coupled to the first and the second distal stent struts, respectively, by stitching.

32. The prosthetic cardiac valve according to claim 29, wherein the junction of the coupling material is mechanically coupled to the frame at or near the distal peak.

33. The prosthetic cardiac valve according to claim 29, wherein the first strip has length equal to at least 50% of a length of the first distal stent strut.

34. The prosthetic cardiac valve according to claim 33, wherein the length of the first strip is greater than the length of the first distal stent strut.

35. The prosthetic cardiac valve according to claim 29, wherein the second strip has length equal to at least 50% of a length of the second distal stent strut.

36. The prosthetic cardiac valve according to claim 35, wherein the length of the second strip is no more than 100% of the length of the second distal stent strut.

37. The prosthetic cardiac valve according to any one of claims 29-36,
wherein the one of the distal stent cells is a first one of the distal stent cells,
wherein the first one of the distal stent cells is joined at a cell junction to a circumferentially-adjacent second one of the distal stent cells, and
wherein the second strip is mechanically coupled to the cell junction.

38. The prosthetic cardiac valve according to claim 37, wherein the second strip is mechanically coupled to the cell junction by stitching.

39. The prosthetic cardiac valve according to any one of claims 29-36,
wherein the prosthetic cardiac valve further comprises an electrical lead, which is electrically coupled to the electrode, and
wherein the first strip is mechanically coupled to at least a portion of the electrical lead.

40. The prosthetic cardiac valve according to claim 39,
wherein the first strip comprises electrical insulation, and
wherein the first strip electrically insulates the at least a portion of the electrical lead.

41. The prosthetic cardiac valve according to claim 40, wherein the first strip comprises an elongate portion of a printed circuit board (PCB) with which the electrical lead is integral.

42. The prosthetic cardiac valve according to claim 39, further comprising circuitry, which is electrically coupled to the electrode by the electrical lead.

43. The prosthetic cardiac valve according to any one of claims 29-36,
wherein the first and the second strips are outer first and second strips, which are mechanically coupled to radially outer sides of the first and the second distal stent struts, respectively,
wherein the coupling material is shaped so as to further define:
(a) an inner first strip that is mechanically coupled to a radially inner side of the first distal stent strut, and
(b) an inner second strip that is mechanically coupled to a radially inner side of the second distal stent strut,
wherein the junction of the coupling material couples together the outer first strip, the outer second strip, the inner first strip, and the inner second strip, and
wherein the outer first strip, the outer second strip, the inner first strip, and the inner second strip together couple the electrode to the frame at or near the distal peak.

44. The prosthetic cardiac valve according to claim 43, wherein the junction of the coupling material is folded over the distal peak.

45. The prosthetic cardiac valve according to claim 44, wherein the folded junction is mechanically coupled to the frame at or near the distal peak.

46. The prosthetic cardiac valve according to any one of claims 29-45, wherein the frame further comprises one or more delivery-tool-coupling tabs, disposed proximal of the stent cells.

47. A valve prosthesis system comprising the prosthetic cardiac valve according to any one of claims 29-46, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

48. The prosthetic cardiac valve according to any one of claims 29-46,
wherein the prosthetic cardiac valve is a prosthetic aortic valve,
wherein the distal half of the frame is an upstream half of the frame,
wherein the distal peaks are upstream peaks,
wherein the distal ones of the stent cells are upstream ones of the stent cells that are located in the upstream half of the frame and define the respective upstream peaks,
wherein the distal peak of the one of the distal stent cells is an upstream peak of one of the upstream stent cells, and
wherein the first and the second distal stent struts of the one of the distal stent cells are first and second upstream stent struts of the one of the upstream stent cells, which are joined at the upstream peak.

49. The prosthetic cardiac valve according to any one of claims 29-46,
wherein the prosthetic cardiac valve is a prosthetic atrioventricular valve,
wherein the distal half of the frame is a downstream half of the frame,
wherein the distal peaks are downstream peaks,
wherein the distal ones of the stent cells are downstream ones of the stent cells that are located in the downstream half of the frame and define the respective downstream peaks,
wherein the distal peak of the one of the distal stent cells is a downstream peak of one of the downstream stent cells, and
wherein the first and the second distal stent struts of the one of the distal stent cells are first and second downstream stent struts of the one of the downstream stent cells, which are joined at the downstream peak.

50. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells, including a first stent cell shaped so as to define:
two peaks, consisting of a distal peak and a proximal peak,
two lateral nodes, consisting of a left lateral node and a right lateral node,
two left stent struts, consisting of (a) a distal left stent strut joined with the distal peak and the left lateral node, and (b) a proximal left stent strut joined with the proximal peak and the left lateral node, and
two right stent struts, consisting of (a) a distal right stent strut joined with the distal peak and the right lateral node, and (b) a proximal right stent strut joined with the proximal peak and the right lateral node;
a plurality of prosthetic leaflets coupled to the frame so as to allow blood flow in a downstream direction and inhibit blood flow in an upstream direction;
an electronic component, which is disposed at or near one of the peaks; and
coupling material, which is shaped so as to define:
(a) a first strip that is mechanically coupled to at least one of the left stent struts,
(b) a second strip that is mechanically coupled to at least one of the right stent struts, and
(c) a junction, which couples together the first and the second strips,
such that the first and the second strips together couple the electronic component to the frame at or near the one of the peaks.

51. The prosthetic cardiac valve according to claim 50, wherein the prosthetic cardiac valve comprises circuitry, which comprises the electronic component, and which is disposed at or near the one of the peaks.

52. The prosthetic cardiac valve according to claim 50, wherein the electronic component comprises an electrode.

53. The prosthetic cardiac valve according to claim 50, wherein the electronic component comprises an energy storage module.

54. The prosthetic cardiac valve according to claim 50, wherein the first and the second strips together couple the electronic component to the frame at least partially outside the first stent cell at or near the one of the peaks.

55. The prosthetic cardiac valve according to claim 50, wherein the first and the second strips are mechanically coupled to the at least one of the left stent struts and the at least one of the right stent struts, respectively, by stitching.

56. The prosthetic cardiac valve according to claim 50, wherein the junction of the coupling material is mechanically coupled to the frame at or near the one of the peaks.

57. The prosthetic cardiac valve according to claim 50, wherein the first strip has length equal to at least 50% of a length of the at least one of the left stent struts.

58. The prosthetic cardiac valve according to claim 57, wherein the length of the first strip is greater than the length of the at least one of the left stent struts.

59. The prosthetic cardiac valve according to claim 50, wherein the second strip has length equal to at least 50% of a length of the at least one of the right stent struts.

60. The prosthetic cardiac valve according to claim 59, wherein the length of the second strip is greater than the length of the at least one of the right stent struts.

61. The prosthetic cardiac valve according to claim 50, wherein the first strip is mechanically coupled to the left lateral node.

62. The prosthetic cardiac valve according to claim 61, wherein the first strip is mechanically coupled to the left lateral node by stitching.

63. The prosthetic cardiac valve according to claim 50, wherein the second strip is mechanically coupled to the right lateral node.

64. The prosthetic cardiac valve according to claim 63, wherein the second strip is mechanically coupled to the right lateral node by stitching.

65. The prosthetic cardiac valve according to any one of claims 50-64,
wherein the prosthetic cardiac valve further comprises an electrical lead, which is electrically coupled to the electronic component, and
wherein the first strip is mechanically coupled to at least a portion of the electrical lead.

66. The prosthetic cardiac valve according to claim 65,
wherein the first strip comprises electrical insulation, and
wherein the first strip electrically insulates the at least a portion of the electrical lead.

67. The prosthetic cardiac valve according to claim 66, wherein the first strip comprises an elongate portion of a printed circuit board (PCB) with which the electrical lead is integral.

68. The prosthetic cardiac valve according to any one of claims 50-67, wherein the frame further comprises one or more delivery-tool-coupling tabs, disposed proximal of the stent cells.

69. A valve prosthesis system comprising the prosthetic cardiac valve according to any one of claims 50-68, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

70. The prosthetic cardiac valve according to any one of claims 50-68,
wherein the prosthetic cardiac valve is a prosthetic aortic valve,
wherein the distal peak and the proximal peak are an upstream peak and a downstream peak, respectively,
wherein the distal left stent strut is an upstream left stent strut,
wherein the proximal left stent strut is a downstream left stent strut,
wherein the distal right stent strut is an upstream right stent strut, and
wherein the proximal right stent strut is a downstream right stent strut.

71. The prosthetic cardiac valve according to any one of claims 50-68,
wherein the prosthetic cardiac valve is a prosthetic atrioventricular valve,
wherein the distal peak and the proximal peak are a downstream peak and an upstream peak, respectively,
wherein the distal left stent strut is a downstream left stent strut,
wherein the proximal left stent strut is an upstream left stent strut,
wherein the distal right stent strut is a downstream right stent strut, and
wherein the proximal right stent strut is an upstream right stent strut.

72. A prosthetic cardiac valve, which is configured to be delivered to a native cardiac valve of a patient in a constrained delivery configuration, and which comprises:
a frame, which defines a central longitudinal axis when the prosthetic cardiac valve is in an expanded deployment configuration, and which comprises interconnected stent struts arranged so as to define interconnected stent cells;
a plurality of prosthetic leaflets coupled to the frame so as to allow blood flow in a downstream direction and inhibit blood flow in an upstream direction when the prosthetic cardiac valve is in the expanded deployment configuration;
circuitry, which is mechanically coupled to the frame;
an electrode, which is mechanically coupled to the frame;
a printed circuit board (PCB), which is shaped so as to define an elongate portion; and
an electrical lead, which electrically couples the electrode to the circuitry, and which is integral with the elongate portion of the PCB,
wherein the elongate portion of the PCB is mechanically coupled to some of the interconnected stent struts of the frame.

73. The prosthetic cardiac valve according to claim 72, wherein the electrical lead is encased in the elongate portion of the PCB.

74. The prosthetic cardiac valve according to claim 72, wherein the elongate portion of the PCB has an undulating shape that generally runs along the interconnected stent struts.

75. The prosthetic cardiac valve according to claim 72, wherein the elongate portion of the PCB is shaped so as to follow a path of the interconnected stent struts.

76. The prosthetic cardiac valve according to claim 72, wherein the elongate portion of the PCB has a same general shape as the interconnected stent struts.

77. The prosthetic cardiac valve according to claim 72, wherein a length of the elongate portion of the PCB, measured in a straight line between endpoints of the elongate portion, equals 50%-100% of a length of the frame, measured parallel to the central longitudinal axis of the frame.

78. The prosthetic cardiac valve according to claim 72, wherein a length of the elongate portion of the PCB, measured in a straight line between endpoints of the elongate portion, equals 150%-1000% of a greatest dimension of the circuitry portion of the PCB.

79. The prosthetic cardiac valve according to claim 72, wherein a length of the elongate portion of the PCB, measured in a straight line between endpoints of the elongate portion, is 0.5-6 cm.

80. The prosthetic cardiac valve according to claim 72, wherein a portion of the elongate portion between the circuitry portion of the PCB and the electrode has a length equal to 50%-100% of a length of the frame, measured parallel to the central longitudinal axis of the frame.

81. The prosthetic cardiac valve according to claim 72, wherein a portion of the elongate portion between the circuitry portion of the PCB and the electrode has a length equal to 150%-1000% of a greatest dimension of the circuitry portion of the PCB.

82. The prosthetic cardiac valve according to claim 72, wherein a portion of the elongate portion between the circuitry portion of the PCB and the electrode has a length of 0.5-6 cm.

83. The prosthetic cardiac valve according to claim 72, wherein the elongate portion of the PCB has a width, perpendicular to a thickness of the PCB, of 0.4-1.5 mm.

84. The prosthetic cardiac valve according to claim 72, wherein the elongate portion of the PCB has a width, perpendicular to a thickness of the PCB, equal to 20%-120% of a shortest dimension of the circuitry portion of the PCB perpendicular to a thickness of the circuitry portion.

85. The prosthetic cardiac valve according to claim 72, wherein the electrode is mechanically coupled to the frame at or near a distal peak of one of distal-most ones of the stent cells.

86. The prosthetic cardiac valve according to claim 72, wherein the stent struts and the elongate portion of the PCB are rectangular in cross section taken perpendicular to respective longitudinal axes of the stent struts and the elongate portion.

87. The prosthetic cardiac valve according to claim 72, wherein a ratio of a thickness of the stent struts to a thickness of the electrical lead is 5-15.

88. The prosthetic cardiac valve according to claim 72, wherein a ratio of a thickness of the stent struts to a thickness of the elongate portion of the PCB is 2-5.

89. The prosthetic cardiac valve according to any one of claims 72-88, wherein the circuitry comprises (a) a circuitry portion of the PCB distinct from the elongate portion of the PCB, (b) tracks of the PCB, (c) conductive pads of the PCB, and (d) electronic components coupled to the PCB.

90. The prosthetic cardiac valve according to claim 89,
wherein the circuitry portion of the PCB is a first circuitry portion of the PCB, and
wherein the PCB is shaped so as to define:
a second circuitry portion, comprising one or more electronic components, and
an elongate circuitry-connecting portion, which connects the first circuitry portion to the second circuitry portion, and which comprises an electrical lead that is integral with the elongate circuitry-connecting portion.

91. The prosthetic cardiac valve according to claim 90, wherein the elongate circuitry-connecting portion is oriented circumferentially around a circumferential portion of the frame.

92. The prosthetic cardiac valve according to claim 90, wherein the one or more electronic components of the second circuitry portion comprise an energy storage module.

93. The prosthetic cardiac valve according to claim 89, wherein the circuitry portion of the PCB is an end portion of the PCB.

94. The prosthetic cardiac valve according to claim 89, wherein the elongate portion of the PCB extends directly from the circuitry portion of the PCB.

95. The prosthetic cardiac valve according to claim 89, wherein the elongate portion of the PCB is integral with the circuitry portion the PCB.

96. The prosthetic cardiac valve according to claim 95, wherein the electrical lead is fabricated as a track of the elongate portion of the PCB in connection with one or more of the tracks of the PCB are that part of the circuitry.

97. The prosthetic cardiac valve according to any one of claims 72-88,
wherein the elongate portion of the PCB is mechanically coupled to some of the interconnected stent struts of the frame by suturing using sutures, and
wherein the elongate portion of the PCB is shaped so as to define a plurality of protrusions along the elongate portion, which inhibit the sutures from sliding along the elongate portion, such that the sutures fix the elongate portion of the PCB securely to the stent struts.

98. The prosthetic cardiac valve according to claim 97, wherein the protrusions protrude laterally from the elongate portion of the PCB in a plane defined by the PCB.

99. The prosthetic cardiac valve according to claim 98, wherein an average distance of lateral protrusion of the protrusions beyond non-protruding portions of the elongate portion, in a single direction, equals 20%-100% of widths of the elongate portion of the PCB at respective locations of the protrusions along the elongate portion, the average distance and the widths measured in the plane defined by the PCB.

100. The prosthetic cardiac valve according to any one of claims 72-88, wherein the elongate portion of the PCB is bifurcated, so as to define a main elongate portion and two or more bifurcation elongate portions.

101. The prosthetic cardiac valve according to claim 100, wherein the electrical lead is bifurcated, so as to define a main portion and two or more bifurcation portions integral with respective bifurcation elongate portions of the elongate portion of the PCB.

102. The prosthetic cardiac valve according to claim 100, wherein the electrical lead is one of a plurality of electrical leads, which are partially integral with the main elongate portion of the elongate portion of the PCB, and partially integral with respective bifurcation elongate portions of the elongate portion of the PCB.

103. The prosthetic cardiac valve according to any one of claims 72-102, wherein the prosthetic cardiac valve is a prosthetic aortic valve.

104. The prosthetic cardiac valve according to claim 103, wherein the circuitry is mechanically coupled to the frame downstream of the prosthetic leaflets, and the electrode is mechanically coupled to the frame upstream of the prosthetic leaflets.

105. The prosthetic cardiac valve according to any one of claims 72-102, wherein the prosthetic cardiac valve is a prosthetic atrioventricular valve.

106. The prosthetic cardiac valve according to claim 105, wherein the circuitry is mechanically coupled to the frame upstream of the prosthetic leaflets, and the electrode is mechanically coupled to the frame downstream of the prosthetic leaflets.

107. A prosthetic cardiac valve, which is configured to be delivered to a native cardiac valve of a patient in a constrained delivery configuration, and which comprises:
a frame, which defines a central longitudinal axis when the prosthetic cardiac valve is in the constrained delivery configuration, and which comprises interconnected stent struts arranged so as to define interconnected stent cells;
a plurality of prosthetic leaflets coupled to the frame so as to allow blood flow in a downstream direction and inhibit blood flow in an upstream direction when the prosthetic cardiac valve is in an expanded deployment configuration; and
an antenna, which is mechanically coupled to the frame, and which comprises:
a magnetic core; and
one or more coils, which are wound about the magnetic core,
wherein, at at least one location along a length of the magnetic core, a planar space bound by an outer perimeter of the magnetic core, in a plane perpendicular to the central longitudinal axis of the frame, has:
(a) a shorter dimension, which is measured along a ray that (i) radiates radially outward from the central longitudinal axis and (ii) intersects a centroid defined by the planar space bound by the outer perimeter, and
(b) a longer dimension, which is measured perpendicular to the shorter dimension in the plane, and is at least 150% of the shorter dimension.

108. The prosthetic cardiac valve according to claim 107, wherein the longer dimension is at least 175% of the shorter dimension.

109. The prosthetic cardiac valve according to claim 108, wherein the longer dimension is at least 200% of the shorter dimension.

110. The prosthetic cardiac valve according to claim 108, wherein the longer dimension is no more than 400% of the shorter dimension.

111. The prosthetic cardiac valve according to claim 107, wherein the longer dimension is no more than 350% of the shorter dimension.

112. The prosthetic cardiac valve according to claim 107, wherein the central longitudinal axis intercepts the plane defined by outer perimeter outside the planar space.

113. The prosthetic cardiac valve according to claim 107,
wherein an external surface of the magnetic core is shaped so as to define an axially-oriented groove,
wherein at least one of the one or more coils comprises a wire, and
wherein a straight portion of the wire is disposed at least partially within the axially-oriented groove, so as to pass from a first axial end to a second axial end of the at least one of the coils.

114. The prosthetic cardiac valve according to claim 107, further comprising:
a cathode and an anode, which are mechanically coupled to the frame; and
circuitry, which is electrically coupled to the cathode, the anode, and the one or more coils.

115. The prosthetic cardiac valve according to any one of claims 107-114, wherein a radially-outward portion of the outer perimeter of the magnetic core, which includes a point on the outer perimeter farthest from the central longitudinal axis, is concavely curved with respect to the central longitudinal axis.

116. The prosthetic cardiac valve according to claim 115, wherein the radially-outward portion of the outer perimeter of the magnetic core has a greatest radius of curvature of 1-5 mm.

117. The prosthetic cardiac valve according to claim 115, wherein the radially-outward portion of the outer perimeter of the magnetic core has a greatest radius of curvature of 0.3-1.6 times the longer dimension.

118. The prosthetic cardiac valve according to claim 115, wherein a radially-inward portion of the outer perimeter, which includes a point on the outer perimeter closest to the central longitudinal axis, is flat.

119. The prosthetic cardiac valve according to claim 115, wherein a radially-inward portion of the outer perimeter, which includes one or more points on the outer perimeter closest to the central longitudinal axis, is concavely curved with respect to the central longitudinal axis.

120. The prosthetic cardiac valve according to claim 119, wherein the radially-inward portion of the outer perimeter has a greatest radius of curvature that is less than a greatest radius of curvature of the radially-outward portion of the outer perimeter.

121. The prosthetic cardiac valve according to claim 119, wherein the curved radially-outward portion of the outer perimeter includes an arcuate portion of a circle.

122. The prosthetic cardiac valve according to claim 121, wherein the arcuate portion has a measure of 45-180 degrees.

123. The prosthetic cardiac valve according to claim 122, wherein the measure is 60-120 degrees.

124. The prosthetic cardiac valve according to any one of claims 107-114,
wherein the magnetic core is shaped so as to define a cavity, and
wherein the prosthetic cardiac valve further comprises circuitry, which is disposed at least partially within the cavity, and which is electrically coupled to the one or more coils.

125. The prosthetic cardiac valve according to claim 124, wherein the circuitry is disposed entirely within the cavity.

126. The prosthetic cardiac valve according to claim 124, wherein the magnetic core has an average wall thickness surrounding the cavity of 100-500 microns.

127. The prosthetic cardiac valve according to claim 124, wherein the magnetic core has an average wall thickness surrounding the cavity equal to 0.05-0.4 times the shorter dimension.

128. The prosthetic cardiac valve according to any one of claims 107-114,
wherein the magnetic core is elongate,
wherein the one or more coils comprise:
first, second, and third coils, wound around the elongate magnetic core such that:
the first coil encircles a first-coil longitudinal axis that coincides with a central longitudinal axis of the elongate magnetic core,
the second coil encircles a second-coil longitudinal axis that is perpendicular to the first-coil longitudinal axis, and
the third coil encircles a third-coil longitudinal axis that is perpendicular to the first-coil longitudinal axis and to the second-coil longitudinal axis,
wherein the second and the third coils cross each other at both longitudinal ends of the elongate magnetic core,
wherein the second coil has two longer sides and two shorter sides, and
wherein the two longer sides are parallel to the central longitudinal axis of the elongate magnetic core, or define an angle of less than 10 degrees with respect to the central longitudinal axis of the elongate magnetic core.

129. The prosthetic cardiac valve according to claim 128,
wherein the third coil has two longer sides and two shorter sides, and
wherein the two longer sides of the third coil are parallel to the central longitudinal axis of the elongate magnetic core, or define an angle of less than 10 degrees with respect to the central longitudinal axis of the elongate magnetic core.

130. The prosthetic cardiac valve according to claim 128, wherein the two longer sides cross the first coil at a plurality of first locations, and define angles of 75-90 degrees with the first coil at each of the plurality of first locations.

131. The prosthetic cardiac valve according to claim 130,
wherein the third coil has two longer sides and two shorter sides, and
wherein the two longer sides of the third coil cross the first coil at a plurality of second locations, and define angles of 75-90 degrees with the first coil at each of the plurality of second locations.

132. The prosthetic cardiac valve according to claim 128,
wherein an external surface of the elongate magnetic core is shaped so as to define an axially-oriented groove,
wherein the first coil comprises a wire, and
wherein a straight portion of the wire is disposed at least partially within the axially-oriented groove, so as to pass from a first axial end to a second axial end of the first coil.

133. The prosthetic cardiac valve according to any one of claims 107-132, wherein the frame further comprises one or more delivery-tool-coupling tabs, disposed proximal of the stent cells.

134. A valve prosthesis system comprising the prosthetic cardiac valve according to any one of claims 107-133, the valve prosthesis system further comprising a delivery system, which comprises:
a delivery sheath, in which the prosthetic cardiac valve is disposed when in the constrained delivery configuration; and
a user-control handle, which is disposed at a proximal end portion of the delivery sheath, wherein an opposite, free end portion of the delivery sheath is a distal end portion of the delivery sheath.

135. The prosthetic cardiac valve according to any one of claims 107-133, wherein the prosthetic cardiac valve is a prosthetic aortic valve, and wherein the antenna is mechanically coupled to the frame downstream of the prosthetic leaflets.

136. The prosthetic cardiac valve according to any one of claims 107-133, wherein the prosthetic cardiac valve is a prosthetic atrioventricular valve, and wherein the antenna is mechanically coupled to the frame upstream of the prosthetic leaflets.

137. The prosthetic cardiac valve according to any one of claims 107-133, wherein the antenna is mechanically coupled to the frame proximal of the prosthetic leaflets.

138. A valve prosthesis system comprising the prosthetic cardiac valve according to any one of claims 107-137, the valve prosthesis system further comprising an external unit,
wherein the one or more coils are one or more prosthetic-valve coils, and
wherein the external unit is configured to be disposed outside a body of the patient, and comprises:
an energy-transmission coil; and
external-unit control circuitry, which is configured to drive the energy-transmission coil to wirelessly transfer energy to at least one of the one or more prosthetic-valve coils by inductive coupling.

139. Apparatus comprising an implantable medical device, which comprises:
an antenna, which comprises:
a magnetic core, which is shaped so as to define a cavity; and
one or more coils, which are wound about the magnetic core; and
circuitry, which is disposed at least partially within the cavity, and which is electrically coupled to the one or more coils.

140. The apparatus according to claim 139, wherein the circuitry is disposed entirely within the cavity.

141. The apparatus according to claim 139, wherein the magnetic core has an average wall thickness surrounding the cavity of 100-500 microns.

142. The apparatus according to claim 139, further comprising a cathode and an anode, which are electrically coupled to the circuitry.

143. The apparatus according to any one of claims 139-142, wherein the implantable medical device comprises a prosthetic cardiac valve, which is configured to be delivered to a native cardiac valve of a patient in a constrained delivery configuration, and which comprises:
a frame, which comprises interconnected stent struts arranged so as to define interconnected stent cells; and
a plurality of prosthetic leaflets coupled to the frame so as to allow blood flow in a downstream direction and inhibit blood flow in an upstream direction when the prosthetic cardiac valve is in an expanded deployment configuration,
wherein the antenna is mechanically coupled to the frame.

144. The apparatus according to claim 143, wherein the antenna is mechanically coupled to the frame proximal of the prosthetic leaflets.

145. The apparatus according to claim 143,
wherein the frame defines a central longitudinal axis when the prosthetic cardiac valve is in the constrained delivery configuration, and
wherein, at at least one location along a length of the magnetic core, a planar space bound by an outer perimeter of the magnetic core, in a plane perpendicular to the central longitudinal axis of the frame, has:
(a) a shorter dimension, which is measured along a ray that (i) radiates radially outward from the central longitudinal axis and (ii) intersects a centroid defined by the planar space bound by the outer perimeter, and
(b) a longer dimension, which is measured perpendicular to the shorter dimension in the plane, and is at least 150% of the shorter dimension.

146. A valve prosthesis system comprising the apparatus according to claim 143, the valve prosthesis system further comprising an external unit,
wherein the one or more coils are one or more medical-device coils, and
wherein the external unit is configured to be disposed outside a body of the patient, and comprises:
an energy-transmission coil; and
external-unit control circuitry, which is configured to drive the energy-transmission coil to wirelessly transfer energy to at least one of the one or more medical-device coils by inductive coupling.

147. A prosthetic cardiac valve system comprising a prosthetic cardiac valve, which is configured to be delivered to a native cardiac valve of a heart of a patient in a constrained delivery configuration, and which comprises:
a frame;
a plurality of prosthetic leaflets coupled to the frame so as to allow blood flow in a downstream direction and inhibit blood flow in an upstream direction when the prosthetic cardiac valve is in an expanded deployment configuration;
electrodes, which include one or more cathodes and one or more anodes, which are mechanically coupled to the frame; and
circuitry, which is electrically coupled to the electrodes, and which is configured to apply pacing to the heart using a subset of the electrodes that includes fewer than all of the electrodes, at least one of the one or more cathodes, and at least one of the one or more anodes.

148. The prosthetic cardiac valve system according to claim 147, wherein the prosthetic cardiac valve system is configured to select the subset of the electrodes by separately activating different combinations of the electrodes at different times, and selecting the subset of the electrodes that provides most effective pacing.

149. The prosthetic cardiac valve system according to claim 148, wherein the prosthetic cardiac valve is configured to sense an ECG of the heart, and wherein the prosthetic cardiac valve system is configured to select the subset of the electrodes based on the ECG sensed when separately activating the different combinations of the electrodes at the different times.

150. The prosthetic cardiac valve system according to claim 148, wherein the prosthetic cardiac valve system is configured to select the subset of the electrodes by separately activating different combinations of the electrodes before the circuitry applies each pulse of the pacing.

151. The prosthetic cardiac valve system according to claim 148, wherein the circuitry of the prosthetic cardiac valve is configured to select the subset of the electrodes.

152. The prosthetic cardiac valve system according to claim 148, wherein the circuitry is prosthetic-aortic-valve circuitry, and wherein the prosthetic cardiac valve system comprises an external control unit, which comprises external circuitry that is configured to select the subset of the electrodes.

153. The prosthetic cardiac valve system according to any one of claims 147-152, wherein the prosthetic cardiac valve is a prosthetic aortic valve.

154. The prosthetic cardiac valve system according to any one of claims 147-152, wherein the prosthetic cardiac valve is a prosthetic atrioventricular valve.

155. A valve prosthesis system for use with a guidewire, the valve prosthesis system comprising:
(i) a prosthetic cardiac valve, which is configured to be delivered to a native cardiac valve of a patient in a constrained delivery configuration using the guidewire, and which comprises: (a) a frame; (b) a plurality of prosthetic leaflets coupled to the frame; (c) a cathode and an anode, which are mechanically coupled to the frame; and (d) an antenna, which comprises one or more prosthetic-valve coils, and which is in electrical communication with the cathode and the anode; and
(ii) an external unit, which is configured to be disposed outside a body of the patient, and which comprises:
(a) a housing, which is shaped so as to define a guidewire-receiving channel;
(b) a rapid-pacing user control;
(c) an energy-transmission coil; and
(d) external-unit control circuitry, which is configured to:
drive the energy-transmission coil to wirelessly transfer energy to at least one of the one or more prosthetic-valve coils by inductive coupling, and
only upon activation of the rapid-pacing user control and when the guidewire is disposed within the guidewire-receiving channel of the housing, drive the prosthetic cardiac valve to apply rapid pacing using the cathode and the anode.

156. The valve prosthesis system according to claim 155, wherein the prosthetic cardiac valve is a prosthetic aortic valve.

157. The valve prosthesis system according to claim 155, wherein the prosthetic cardiac valve is a prosthetic atrioventricular valve.

158. Apparatus comprising an implantable medical device, which comprises:
an antenna, which comprises:
an elongate core; and
first, second, and third coils, wound around the elongate core such that:
the first coil encircles a first-coil longitudinal axis that coincides with a central longitudinal axis of the elongate core,
the second coil encircles a second-coil longitudinal axis that is perpendicular to the first-coil longitudinal axis, and
the third coil encircles a third-coil longitudinal axis that is perpendicular to the first-coil longitudinal axis and to the second-coil longitudinal axis,
wherein the second and the third coils cross each other at both longitudinal ends of the elongate core,
wherein the second coil has two longer sides and two shorter sides, and
wherein the two longer sides are parallel to the central longitudinal axis of the elongate core, or define an angle of less than 10 degrees with respect to the central longitudinal axis of the elongate core.

159. The apparatus according to claim 158,
wherein the third coil has two longer sides and two shorter sides, and
wherein the two longer sides of the third coil are parallel to the central longitudinal axis of the elongate core, or define an angle of less than 10 degrees with respect to the central longitudinal axis of the elongate core.

160. The apparatus according to claim 158, wherein the two longer sides cross the first coil at a plurality of first locations, and define angles of 75-90 degrees with the first coil at each of the plurality of first locations.

161. The apparatus according to claim 160,
wherein the third coil has two longer sides and two shorter sides, and
wherein the two longer sides of the third coil cross the first coil at a plurality of second locations, and define angles of 75-90 degrees with the first coil at each of the plurality of second locations.

162. The apparatus according to claim 158, wherein an external surface of the elongate core is shaped so as to define an axially-oriented groove, wherein the first coil comprises a wire, and wherein a straight portion of the wire is disposed at least partially within the axially-oriented groove, so as to pass from a first axial end to a second axial end of the first coil.

163. A prosthetic cardiac valve system comprising a prosthetic cardiac valve, which is configured to be delivered to a native cardiac valve of a heart of a patient in a constrained delivery configuration, and which comprises:
a frame, which comprises interconnected stent cells, which include distal stent cells that are located in a distal half of the frame and are shaped so as to define respective distal peaks;
a plurality of prosthetic leaflets coupled to the frame so as to allow blood flow in a downstream direction and inhibit blood flow in an upstream direction when the prosthetic cardiac valve is in an expanded deployment configuration;
electrodes, which include a plurality of distal electrodes mechanically coupled to the frame at or near respective ones of the distal peaks; and
circuitry, which is electrically coupled to the electrodes, and which is configured to apply pacing to the heart by activating one or more of the distal electrodes as one or more anodes and one or more of the other distal electrodes as one or more cathodes.

164. The prosthetic cardiac valve system according to claim 163, wherein the distal electrodes are mechanically coupled to the frame at or within 8 mm of the respective ones of the distal peaks.

165. The prosthetic cardiac valve system according to claim 163, wherein the distal stent cells are distal-most ones of the stent cells.

166. The prosthetic cardiac valve system according to claim 163, wherein the distal peaks are respective upstream peaks, and wherein the distal electrodes are upstream electrodes that are mechanically coupled to the frame at or near respective ones of the upstream peaks.

167. The prosthetic cardiac valve system according to any one of claims 163-166, wherein the prosthetic cardiac valve is a prosthetic aortic valve.

168. The prosthetic cardiac valve system according to any one of claims 163-166, wherein the prosthetic cardiac valve is a prosthetic atrioventricular valve.
